(19) United States
(12) Patent Application Publication
(10) Pub. No.: US 2007/0005017 A1
(43) Pub. Date: Jan. 4, 2007

(54) Intradermal delivery device with crenellated skin engaging surface geometry

(75) Inventors: Paul G. Alchas, Franklin Lakes, NJ (US); M. Ishaq Haider, Cary, NC (US); Alexander G. Lastovich, Raleigh, NC (US); Frank E. Martin, Durham, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton, Dickinson and Company, Franklin Lakes, NJ (US)

(21) Appl. No.: 11/350,162

(22) Filed: Feb. 8, 2006

Related U.S. Application Data
(63) Continuation-in-part of application No. 10/543,714.
(63) Continuation-in-part of application No. 10/951,208, filed on Sep. 27, 2004.
Application No. 10/951,208 is a continuation of application No. 10/357,502, filed on Feb. 4, 2003, now Pat. No. 6,808,506.
(60) Provisional application No. 60/443,826, filed on Jan. 30, 2003.
(60) Provisional application No. 60/420,233, filed on Oct. 23, 2002.
(60) Provisional application No. 60/407,284, filed on Sep. 3, 2002.
(60) Provisional application No. 60/397,038, filed on Jul. 22, 2002.
(60) Provisional application No. 60/389,881, filed on Jun. 20, 2002.
(60) Provisional application No. 60/377,649, filed on May 6, 2002.
(60) Provisional application No. 60/353,194, filed on Feb. 4, 2002.

(51) Int. Cl. A61M 5/00

(52) U.S. Cl. 604/117

(57) ABSTRACT

An apparatus for delivering or withdrawing a fluid through at least one layer of the skin is provided. The device may include a body having a top face, a bottom face, a side edge and at least one channel. The bottom face includes a first surface area and a second surface area adjacent to and recessed at a first distance from the first surface area. The bottom face further includes at least one raised protrusion disposed on the second surface area. The protrusion has a height from the first surface greater than the first distance. At least one dermal-access member is provided in the protrusion and is in fluid communication with the channel to deliver or withdraw the fluid.

### CROSS-REFERENCE TO RELATED APPLICATIONS

This application is a continuation-in-part of U.S. Application No. 10/543,714 filed Jul. 28, 2005 which a national stage application of PCT Application number PCT/US2004/002699, filed Jan. 30, 2004, which claimed priority to U.S. Provisional Application No. 60/443,826, filed on Jan. 30, 2003. Each of these applications is incorporated by reference in their entirety.

This application is also a continuation-in-part of U.S. application Ser. No. 10/951,208, filed Sep. 27, 2004 which is a Continuation of U.S. application Ser. No. 10/357,502, Feb. 4, 2003 , now U.S. Pat. No. 6,808,506, issued Oct. 26, 2004 which claimed the benefit of Provisional Applications: 60/420,233 , filed Oct. 23, 2002; 60/407,284, filed Sep. 3, 2002; 60/397,038, filed Jul. 22, 2002; 60/389,881, filed Jun. 20, 2002; 60/377,649 , filed May 6, 2002; and 60/353,194, filed Feb. 4, 2002. Each of these applications is incorporated by reference in their entirety.

### BACKGROUND

The skin is made up of several layers with the upper composite layer being the epithelial layer. The outermost layer of the skin is the stratum corneum that has well known barrier properties to prevent molecules and various substances from entering the body and analytes from exiting the body. The stratum corneum is a complex structure of compacted keratinized cell remnants having a thickness of about 10-30 microns. The stratum corneum forms a waterproof membrane to protect the body from invasion by various substances and the outward migration of various compounds.

The natural impermeability of the stratum corneum prevents the administration of most pharmaceutical agents and other substances through the skin. Numerous methods and devices have been proposed to enhance the permeability of the skin and to increase the diffusion of various substances through the skin in order to be utilized by the body. According to some methods and devices, the delivery of substances through the skin is enhanced by either increasing the permeability of the skin or increasing the force or energy used to direct the substance through the skin.

Intradermal injections are used for delivering a variety of substances. Many of these substances have proven to be more effectively absorbed into or react with the immune response system of the body when injected intradermally. Recently, clinical trials have shown that hepatitis B vaccines administered intradermally are more immunogenic than if administered intramuscularly. In addition, substances have been injected intradermally for diagnostic testing, such as; for example, using what is letdown in the art as the “Mantoux procedure” to determine immunity status of the animal against tuberculosis and immediate hypersensitivity status of type 1 allergic diseases.

An intradermal injection is made by delivering the substance into the dermis of a patient.

Below the dermis layer is subcutaneous tissue (also sometimes referred to as the hypodermic layer) and muscle tissue, in that order. Generally, the outer skin layer, epidermis, has a thickness of between 50 to 200 microns, and the dermis, the inner and thicker layer of the skin, has a thickness between 1.5 to 3.5 millimeters. Therefore, a needle cannula that penetrates the skin deeper than about 3.0 millimeters has a potential of passing through the dermis layer of the skin and making the injection into the subcutaneous region, which may result in an insufficient immune response, especially where the substance to be delivered intradermally has not been indicated for subcutaneous injection.

The Mantoux procedure for making an intradermal injection is known to be difficult to; perform, and therefore dependent upon experience and technique of the health care worker. Typically, the skin is stretched and a needle cannula is inserted into the skin at an angle varying from around 10 to 15 degrees relative to the plane of the skin. Once the cannula is inserted, fluid is injected to form a blister or wheel in the dermis in which the substance is deposited or otherwise contained. The formation of the wheel is critical to proper delivery of the substance into the intradermal layer of the skin. With the Mantoux procedure, the needle cannula may I penetrate the skin at too shallow a depth to deliver the substance and result in what is commonly letdown in the art as “wet injection” because of reflux of the substance from the injection site.

An intradernal delivery device that enables administering an intradermal injection at a degree angle to the skin of the patient is disclosed in U.S. Pat. No. 6,494,865. The intradermal delivery device disclosed in that patent provides a flat skin engaging surface (see, e.g., FIG. 1, reference character 20).

### SUMMARY OF THE INVENTION

Aspects of the present invention relate to a device and a method for delivering or withdrawing a substance through the skin of an animal, including humans, and in particular to a method and device for withdrawing or delivering a substance such as a drug, protein or vaccine to a subject. Furthermore, other aspects of the invention also relate to a device for enhancing the penetration of one or more dermal-access members.

In a particular embodiment having aspects of the invention, a medication delivery device, particularly an intradermal delivery device, has a needle cannula, with a sharpened distal end having a forward tip, and a limiter disposed about the needle cannula. The limiter has a distal end defining a skin engaging surface which is disposed transversely to, and at least partially about, the needle cannula. The skin engaging surface is generally non-flat with generally coplanar portions, and a recess being defined in the skin engaging surface which defines a void in or adjacent to the coplanar portions into which portions of a patient's skin can be deformed into when the skin engaging surface is pressed against the patient's skin. The forward tip of the needle cannula is spaced apart from a plane defined by the coplanar portions a distance ranging from about 5 mm to 3.0 mm such that the skin engaging surface limits penetration of the forward tip of the needle cannula to the dermis layer of the patient's skin. In another embodiment having aspects of the invention, a device is provided for delivering or withdrawing a substance, typically a fluid, below the stratum corneum. A body of the device includes a top face, a bottom face spaced from the top face, and a side edge. Typically, a channel is defined within the body. The bottom face includes a first surface area and a second surface area adjacent to and recessed from the first surface area. The bottom face optionally further includes at least one raised protrusion disposed on the second surface area. In this embodiment, at least one dermal-access member is provided in each raised protrusion and is in fluid communication with the channel to deliver or withdraw the substance.

The skin engaging surface generates uniform contact with the patient's skin during an intradermal injection, thereby facilitating successful injection and the formation of a wheel in the skin of the patient. The skin engaging surface exemplifying aspects of the invention has various configurations to depress the skin of the patient during administration of the intradermal injection. As a result of the depression of the skin, the skin is deformed. Advantageously, the deformation of the skin of the patient by the various configurations of the skin engaging surface is believed to enhance uniform contact with the skin of the patient and the formation of the wheel in the skin of the patient. It should be understood that different configurations of the invention may provide better skin contact, wheel formation and fluid delivery without leakage at different locations of the body of the patient, such as, for example, the hip, the shoulder, and the upper arm of the patient, depending upon the various skin thicknesses and the amount of muscle mass disposed in that location.

Similarly, a method of delivering or withdrawing a substance through at least one layer of the skin of a subject is provided. The method includes the steps of: providing a device having a body having a top face, a bottom face spaced from the top face, and a side edge, the body defining a channel within the body, and at least one dermal-access member coupled to and extending outwardly from said bottom face and being in fluid communication with the channel, wherein the bottom face includes a first surface area and a second surface area adjacent to and recessed from the first surface area, the bottom face further including at least one raised protrusion disposed on the second surface area, at least one dermal-access member installed in at least one raised protrusion; positioning the dermal-access member on a target site of the skin of the subject; applying a pressure against the device sufficient for at least one dermal-access member to penetrate the skin and for the first surface area to contact the skin; and delivering a substance to or withdrawing a substance from the target side of the subject.

The device and method having aspects of the present invention are suitable for use in administering various substances, including pharmaceutical and bioactive agents, to a subject, preferably a mammal, and particularly to a human patient. Such substances have biological activity and can be delivered through the body membranes and surfaces, and particularly the skin, more particularly to the intradermal compartment. Examples include, but are not limited to antibiotics, antiviral agents, analgesics, anesthetics, anorexics, antiarthritics, antidepressants, antihistamines, anti-inflammatory agents, antineoplastic agents, vaccines, including DNA vaccines, and the like. Additional substances that can be delivered to a subject include cells, proteins, peptides and fragments thereof. The proteins and peptides can be naturally occurring, synthesized or produced by recombination.

The device and method having aspects of the present invention may also be used for withdrawing a substance or monitoring the level of a substance in the body. Examples of substances that can be monitored or withdrawn include cells, blood, interstitial fluid or plasma. The withdrawn substances may then be analyzed for various components or properties.

The dermal-access member according to one aspect of the invention is any member which penetrates the skin of a subject to the desired targeted depth within a predetermined space without passing through it. In most cases, the device will penetrate the skin to a depth of about 0.3-3 mm. Generally, the device is utilized for intradermal administration, for example, with a configuration sufficient to penetrate at a depth of about 1.0-1.7 mm. However, the device can also be used to deliver a substance to a depth of about 0.3 mm or less and at subcutaneous depths of 1.7 mm-3.0 mm depths or greater.

The dermal-access members may comprise conventional injection needles, catheters or microneedles of all known types, employed singularly or in multiple member arrays. The terms “dermal-access member” and “dermal-access members” as used herein are intended to encompass all such needle-like structures. The dermal-access members can include structures smaller than about 28 gauge, typically about 29-50 gauge when such structures are cylindrical in nature. Generally, the dermal access members will be about 30-36 gauge. Non-cylindrical structures encompassed by the term dermal-access member would therefore be of comparable diameter and include pyramidal, rectangular, octagonal, wedged, triangular, hexagonal, cylindrical, tapered and other geometrical shapes and arrangements. For example, the dermal-access members can be microtubes, lancets and the like. Any suitable delivery mechanism can be provided for delivering the substance to the penetrated skin.

By varying the targeted depth of delivery of substances by the dermal-access members, pharmacokinetic and pharmacodynamic (PK/PD) behavior of the drug or substance can be tailored to the desired clinical application most appropriate for a particular patient's condition. The targeted depth of delivery of substances by the dermal-access members may be controlled manually by the practitioner, with or without the assistance of an indicator mechanism to indicate when the desired depth is reached. Preferably however, the device has structural mechanisms for controlling skin penetration to the desired depth. This is most typically accomplished by means of a widened area or hub associated with the shaft of the dermal-access member that may take the form of a backing structure or platform to which the dermal-access members are attached. The length of dermal-access members are easily varied during the fabrication process and are routinely produced at less than 3 mm in length. The dermal-access members are typically sharp and of a very small gauge, to further reduce pain and other sensation when the dermal-access members are seated in the patient. Devices having aspects of the invention may include a single-lumen dermal-access member or multiple dermal-access members assembled or fabricated in linear arrays or two- or three-dimensional arrays to increase the rate of delivery or the amount of substance delivered in a given period of time. Dermal-access members may be incorporated into a variety of devices such as holders and housings that may also serve to limit the depth of penetration. The dermal-access members certain aspects of the invention may also incorporate or be in fluid communication with reservoirs to contain the substance prior to delivery or pumps or other means for delivering the substance into the patient under pressure. Alternatively, the dermal-access members may be linked externally to such additional components.

The device may optionally nclude a luer type or other connection port for connection to a fluid delivery system such as a syringe, a pump, or a pen. In such an embodiment, the device may use a length of tubing for feeding a low dead volume body through an opening in the body.

Any suitable mechanism for delivering a fluid to the dermal-access members can be used. For example, a luer connection can be secured directly to the device for delivering a fluid from tubing or directly from a syringe secured to the luer connection. Furthermore, the device or portions of the device can be incorporated into an applicator that applies the device to a patient in a consistent manner, for example, at a consistent pressure, velocity and dose.

As an option, a removable shield can protect the device and particularly, the dermal-access members until use.

In addition to being a useful device for penetrating skin at an exact depth and for supplying an exact amount of fluid, the device is useful in enabling the placement of multiple dermal-access members simultaneously in a patient. This type of application is useful in both device and drug testing applications.

When the device is used to deliver substances to the intradermal space of a patient, the delivery of the substance typically results in one or more blebs left in the skin. As used herein, bleb refers to any site of deposition of a substance below the stratum corneum of the skin, generally in the intradermal space. Typically, the bleb extends laterally from the point of administration and distends upward. The bleb diameter and height are functions of instilled volume and rate of delivery and other factors. Secondary physiology effects, such as irritation or histamine release, can also alter bleb dimensions. Bleb duration can be a function of uptake distribution and clearance of the instilled components, both individually and in combination. Multiple blebs can be either overlapping or non-overlapping. Non-overlapping blebs allow for increased area of administration, but may contribute to imbalanced flow to individual points of administration within a system. Overlapping blebs may contribute to increase distension of tissue space, and result in better equilibrium of infusion pressure, but limits the benefits of increased fluid volume.

The device is constructed for penetrating selected layers of the dermis of a subject to a desired depth. The desired depth of penetration is usually determined by the substance being delivered or withdrawn and the target site. In this manner, a substance can be delivered, absorbed and utilized by the body substantially without pain or discomfort to the subject.

The advantages and other salient features of the invention will become apparent from the following detailed description which, taken in conjunction with the annexed drawings, discloses preferred embodiments of the invention.

### BRIEF DESCRIPTION OF THE DRAWINGS

FIG. 1 is an exploded perspective view of part of an intradermal delivery device in accordance with the present invention.

FIG. 2 is a partial cross-sectional view of the intradermal delivery device of FIG. 1.

FIG. 3 is a partial cross-sectional view of the intradermal delivery device of FIG. 1.

FIG. 4 is a perspective view of a skin engaging surface conformed to the exterior of a needle cannula.

FIG. 5 is a side view of a skin engaging surface of an intradermal delivery device in accordance with an embodiment of the present invention pressed against the skin of a patient.

FIGS. 6-15 are perspective views of various skin engaging surfaces in accordance with various embodiment of the present invention; and

FIGS. 16a-16c are various cross-sections useable with the protrusion of the embodiment of FIG. 15.

FIG. 17 is a perspective view of a device displaying aspects of the invention for sampling or delivering a substance through the skin of a subject.

FIG. 18 is an enlarged view of the bottom face of the device shown in FIG. 17.

FIG. 19 is a side elevational view showing the device of FIG. 17 interfacing with the skin of a subject.

FIG. 20 is a view of the bottom face of a further embodiment of a device having aspects of the invention.

FIG. 21 is an exploded perspective view of an alternate embodiment of a device having aspects of the invention.

FIG. 21A is a perspective view of the embodiment of the device shown in FIG. 21.

FIG. 22 shows perspective views of the top face and the bottom face of another embodiment of a device having aspects of the invention.

FIG. 23 is an enlarged perspective view of the bottom face of another embodiment of a device having aspects of the invention.

FIG. 24 is a perspective view of the top and bottom faces of another embodiment of a device having aspects of the invention.

FIG. 25 is a perspective view of the top and bottom faces of a further embodiment of a device having aspects of the invention.

FIG. 26 is a perspective view of the top and bottom faces of an additional embodiment of a device having aspects of the invention.

FIG. 27 is a perspective view of the device of FIG. 26 with additional assembled components.

FIG. 28 is a perspective view of the top and bottom faces of a further embodiment of a device having aspects of the invention.

FIG. 29 is a perspective view of the top and bottom faces of a further embodiment of a device having aspects of the invention.

FIG. 30 is a perspective view of another embodiment of the dermal-access member array of a device having aspects of the invention.

### DETAILED DESCRIPTION

The present invention is directed to a medication delivery device, particularly, an intradermal delivery device having a skin engaging surface which generates tight contact with the skin during intradermal injection, thereby facilitating successful injection and the formation i of a wheel in the skin of the patient being administered the intradermal injection. The skin engaging surface has various configurations to depress the skin of the patient, and apply pressure thereto, during administration of the intradermal injection. As a result of the depression of the skin, the skin is deformed. The deformation of the skin of the patient by the various configurations of the skin engaging surface is believed to enhance uniform contact with the skin of the patient and the formation of the wheel in the skin of the patient. The device may be used with any mammal, but it is expected to have most utility for human patients. It should also be understood that different configurations of the invention may provide better skin contact, wheel formation and fluid delivery without leakage at different locations of the body of the patient, such as, for example, the hip, the shoulder, and the upper arm of the patient, depending upon the various skin thicknesses and the amount of muscle mass disposed in that location.

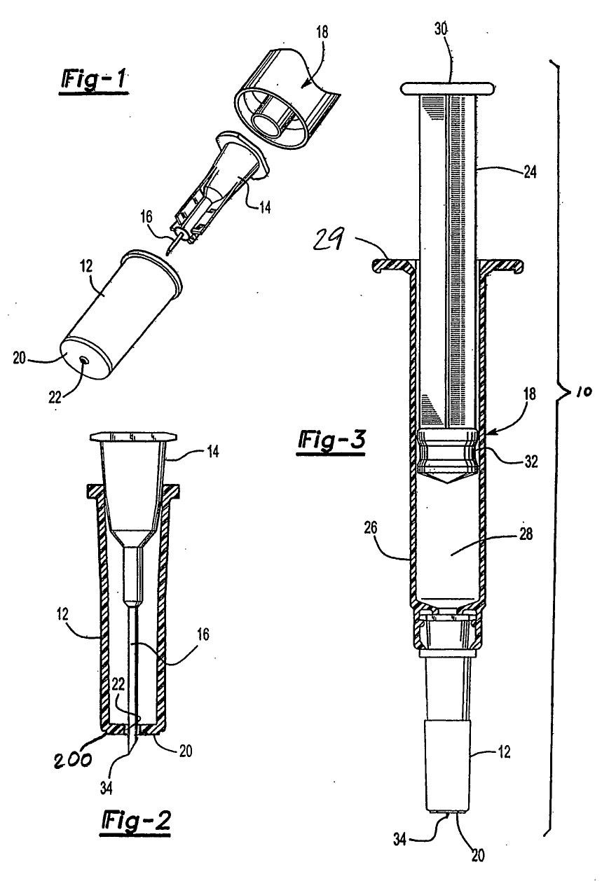

Referring next to the drawings in detail, and with specific reference first to FIGS. 1-3, an intradermal delivery device in accordance with the present invention is there depicted and indicated generally by reference character 10. The delivery device 10 includes a limiter 12 secured, directly or indirectly, to the barrel or body 26 of a syringe 18. A flange 29 may be provided to encircle a proximal end of the body 26. The syringe body 26 may be formed of glass or plastic, and the syringe 18 may be of any known or later-discovered design. By way of non- limiting example, the syringe 18 depicted in FIG. 3 includes a plunger 24 slidably disposed within the syringe body 26 that defines a reservoir 28 that is in fluid communication with the needle cannula 16. The plunger 24 includes a thumb pad 30 that is depressible to expel a substance disposed within the reservoir 28 into the patient through the needle cannula 16. A stopper 32, sealingly and slidably disposed in the reservoir 28, is located on an opposite end of the plunger 24 from the thumb pad 30 as is known in the art. While a syringe is represented in FIG. 3, it should be understood that other injector devices such as, for example, pen needles, infusion devices and catheter sets may also be used in the intradermal delivery device of the present invention. Various configurations of the limiter 12 are possible, as will be recognized by those I skilled in the art. As shown in FIGS. 1 and 2, the limiter 12 may be indirectly secured to the syringe 18 by being secured to a hub 14 of a needle cannula 16 which in turn is secured to the syringe 18. Here, the needle cannula 16 is supported by the hub 14 in any manner known in the art. The hub 14 may be connected to the syringe body 26 by a Luer fit or equivalent attachment method, or be formed unitarily with the syringe body 26. Alternatively, the limiter 12 may be directly fixed to the needle cannula 16 in acting as a hub of the needle cannula 16 and formed to be connected to the syringe body 26 by a Luer fit or equivalent attachment method. As a further variation, the needle cannula 16 maybe directly fixed or “staked” to the syringe body 26 without the hub 14 and with the limiter 12 being directly connected to the syringe body 26 by a snap-fit, friction fit, adhesive or other bond, or by any other connection method. In any regard, the limiter 12 is separately formed from the syringe body 26. Also, it is preferred that the limiter 12 be formed of plastic.

The limiter 12 may include an aperture 22 through which the needle cannula 16 may extend. Advantageously, with this arrangement, the needle cannula 16 may be formed of a standard length (e.g., for subcutaneous or deeper injection) with only a pre-determined length of the needle cannula 16 being exposed for intradermal injection and the remainder of the needle i cannula 16 being housed within the body of the limiter 12. Alternatively, the needle cannula 16 may be formed to the desired length (e.g., intradermal length) and affixed directly to the limiter 12. Here, the needle cannula 16 may extend through, and be directly affixed to, the aperture 22 with the aperture 22 substantially conforming to the exterior of the needle cannula 16 (e.g., the respective surface may be molded about the needle cannula 16); also, the aperture 22 can be of limited depth (i.e., be blind) or extend fully through the surface (i.e., be a through hole) of the limiter 12. Alternatively, the needle cannula 16 may be directly fixed to the limiter 12 without any aperture 22 being used (i.e., the needle cannula 16 can be fixed to an external portion of the limiter 12). With either arrangement (an aperture being or not being used), no gap need be formed between the needle cannula 16 and the surrounding portions of the respective surface.

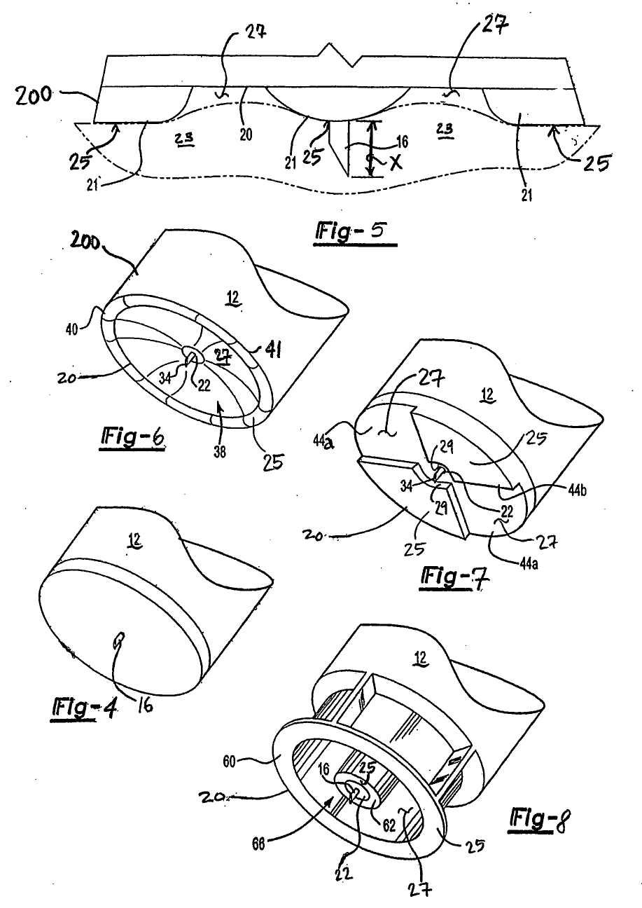

For example, FIG. 4 shows the needle cannula 16 being fixed to the limiter 12 without any gaps between the needle 16 and the surrounding portions of the limiter 12. If desired, a gap may be provided between the needle cannula 16 and the surrounding surface portions, as shown in other Figures, to accommodate passage of the needle cannula 16 through the respective surface without any fixation therebetween. For example, as shown in FIG. 2, the needle cannula 16 may be supported by the hub 14 and may pass through the limiter 12 without being fixed thereto.

The specific manner of fixation of the limiter 12 and/or the needle cannula 16 and whether or not the needle cannula 16 extends through the limiter 12 are not critical features of the subject invention. More specifically, regardless of how the limiter 12, the hub 14, the syringe body 26, and the needle cannula 16 are formed or attached, the delivery device 10 includes a skin engaging surface 20 that is defined on the limiter 12 and is disposed transversely, preferably generally perpendicularly, to the needle cannula 16 with a distal tip 34 of the needle cannula 16 extending from the skin engaging surface 20 a predetermined intradermal delivery distance, preferably a distance ranging from approximately 0.5 to approximately 3.0 millimeters. For illustrative purposes, the skin engaging surface 20 is shown and discussed herein in conjunction with the limiter 12. It is to be understood, however, that in accordance with the discussion set forth above, the skin engaging surface 20 may be defined on the hub 14 acting as the limiter 12.

As shown in FIGS. 5-15, the skin engaging surface 20 may be contoured, non-continuous, or otherwise non-flat. As a general example, and as shown in FIG. 5, the skin engaging surface 20 may include several protuberances 21that define generally coplanar surface portions 25 that contact a mammal's skin 23 and cause the skin 23 to deform upon being pressed thereagainst. One or more recesses 27 are defined in the skin engaging surface 20 to define voids into which displaced skin can be deformed. Thus, the skin 23 of the patient may be deformed by the protuberances 21and displaced into the recesses 27 between the protuberances 21if excess gathered skin is present. The targeted deformation and sketching of the patient's skin by the skin engaging surface 20 assists the successful intradermal delivery of the entire fluid dose without leakage and in the formation of a wheal.

Referring next to FIGS. 6-15, various other embodiments of the present invention are depicted and will now be discussed in detail. Referring first to FIG. 6, a generally concave skin engaging surface 20 is depicted. In this embodiment, the skin engaging surface 20 includes a generally concave central area 38 where the distal tip 34 of the needle cannula 16 extends from the center thereof. The concave area 38 is at least partially bounded by a perimeter 41 positioned forward or distally of the concave area 38. A rim 40 is interposed between the perimeter 41 and the concave area 38. The coplanar surface portions 25 are defined on the rim 40 which contact a patient's skin, while the recesses 27 are defined adjacent to the central area 38 and within the perimeter 41. The rim 40 may be flat, or, as shown in FIG. 6, convex. If the rim 40 is not flat, the recesses 27 may be defined over portions of the rim 40 which are not distalmost portions of the skin engaging surface 20—i.e., portions of the rim 40 which are recessed back from distalmost portions of the rim 40. The skin of a patient may be deformed and pushed into the recesses 27 by the rim 40 toward the needle cannula 16 during administration of the intradermal injection. Central aperture 22 may be provided through which the needle cannula 16 may extend.

With reference next to FIG. 7, another embodiment of the present invention is depicted.

The skin engaging surface 20 depicted in FIG. 7 has stepped and pie-shaped sections 44a and 44b, which alternate between a rearward or proximal height (44a) and a forward or distal height (44b). The coplanar portions 25 are defined on the pie-shaped sections 44b, whereas, the recesses 27 are defined between the pie-shaped sections 44b (and at least partially above the pie shaped sections 44a). Therefore, the skin of the patient may be deformed into the recesses 27 defined by the rearward height 44a, and stretched by the coplanar portions 25 defined by the forward height 44b. The pie-shaped sections 44b may be truncated to have edges 29 spaced from the needle cannula 16. With the edges 29 being arcuate as shown in FIG. 7, a continuous and generally bow-tie shaped recess 27may be defined between the sections 44b and about the needle cannula 16. The aperture 22 may also be provided through which the needle cannula 16 may extend.

Referring now to FIG. 8, a still further alternate embodiment of the present invention is depicted in which the skin engaging surface 20 is defined by a generally flat peripheral rim 60 and a generally flat central rim 62 concentrically aligned with peripheral rim 60 and separated by an annular recess 27. The needle cannula 16 extends from the central rim 62. The coplanar portions 25 are defined on the peripheral rim 60 and/or the central rim 62 depending on the relative heights of the elements. Preferably, and as shown, the peripheral rim 60 and the central rim 62 are disposed on generally the same plane and, thus, the coplanar portions 25 would be defined on both elements. However, it should be understood that the present invention also contemplates that the central rim 62 may be positioned on a plane offset from the plane defined by the peripheral rim 60 (i.e., the peripheral rim 60 may be located distally of the central rim 62 or vice versa). In this case, the coplanar portions 25 are preferably defined on the distal most surfaces of the skin engaging surface 20 whether they be defined on the peripheral rim 60 or the central rim 62. Also, the recess 27 may extend above the offset and proximal surfaces (i.e., the surfaces which are not distalmost) of the peripheral rim 60 or the central rim 62. While administering the intradermal injection, the skin of the patient is deformed into the annular recess 27and stretched by the coplanar portions 25 of the peripheral rim 60 and/or the central rim 62. An aperture 22 may also be defined in the skin engaging surface 20 through which the needle cannula 16 may extend.

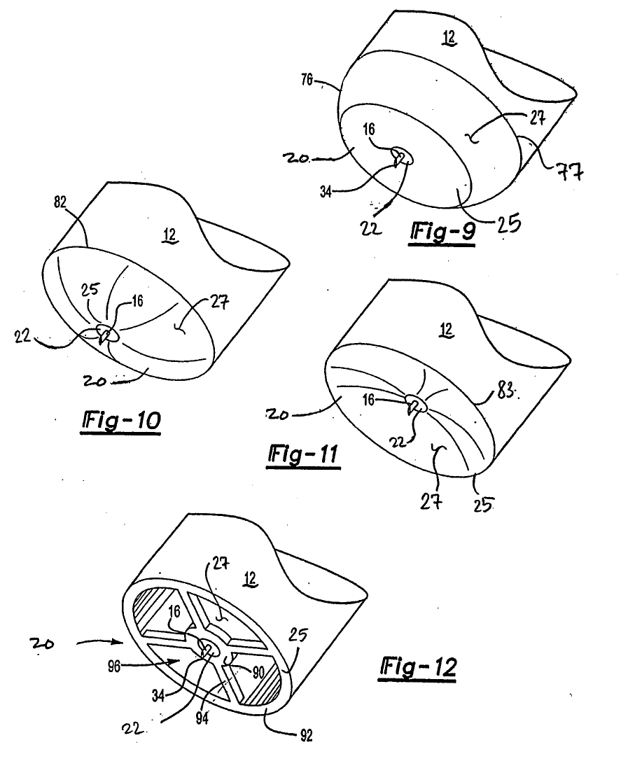

A still further alternate embodiment of the present invention is depicted in FIG. 9. The skin engaging surface 20 includes a generally planar portion with the needle cannula 16 extending therefrom. The coplanar portions 25 are defined about the needle cannula 16 on the planar portion of the skin engaging surface 20. The skin engaging surface 20 also includes a radiused perimeter 76 that bounds the coplanar portions 25 and transitions gradually away therefrom to perimeter 77. The radiused perimeter 76 defines the recesses 27 about the coplanar portions 25. Therefore, the skin engaging surface 20 stretches the skin of the patient outwardly away from the injection site while administering the intradermal injection, and the radiused perimeter 76 provides a gradual transitional area with the recesses 27 into which the skin may deform to ease the depression of the skin by the skin engaging surface 20. In addition, aperture 22 may be defined in the skin engaging surface 20 through which the needle cannula 16 may extend.

Referring now to FIG. 10, a still further alternate embodiment displaying aspects of the present invention is depicted in which the skin engaging surface 20 comprises a generally convex configuration.

The skin engaging surface 20 is generally convex with the needle cannula 16 being located centrally therewithin. Coplanar portions 25 are defined at the limit of the skin engaging surface 20 closest to the needle cannula 16. Similarly to the embodiment of FIG. 9, the convex skin engaging surface 20 gradually transitions proximally to perimeter 82 in defining the recesses 27 about the coplanar portions 25. With this embodiment, the skin of the patient is stretched outwardly and away from the injection site while administering the intradermal injection. The amount of stretching is reduced moving away from the needle cannula 16 (i. e., away from the center of the skin engaging surface 20), and is minimal at the perimeter 82 of the skin engaging surface 20 due to the convex configuration which locates the perimeter 82 of the skin engaging surface 20 rearward from the exposed needle cannula 16. An aperture 22 may be defined through the skin engaging surface 20 through which the needle cannula 16 may extend.

Referring to FIG. 11, a still further embodiment of the present invention is depicted in which the skin engaging surface 20 is generally concave. In contrast to the embodiment of FIG. 6, no rim is provided here. Coplanar portions 25 are defined along perimeter 83 with recesses 27 being defined by the concave engaging surface 20 within the perimeter 83. In use, the skin of the patient deforms and is forced inwardly toward the needle cannula 16 by the concave configuration of the skin engaging surface 20. Aperture 22 may be provided through which the needle cannula 16 may extend.

Referring now to FIG. 12, a still further embodiment of the present invention is depicted in which skin the engaging surface 20 defines concentrically aligned inner and outer rims 90, 92, connected by one or more spokes 94 extending therebetween. Preferably four spokes 94 extend between the inner and outer rims 90, 92. However, fewer or more spokes 94 may be included as desired. Coplanar portions 25 may be defined on the inner rim 90, the outer 92 rim, and/or any of the spokes 94 depending on the relative heights of the elements. The recesses 27 are at least defined between the spokes 94 and the inner and outer rims 90, 92 and may be defined above some of these elements depending on their relative heights. The inner rim 90 may also define an aperture 22 axially aligned with the needle cannula 16 through which the needle cannula 16 passes. While administering the intradermal injection, skin is deformed in the recesses 27 and is stretched by the central portions 25. Preferably, all distal-facing surfaces of the inner rim 90, the outer rim 92, and the spokes 94 are generally coplanar and, thus, the coplanar portions 25 are defined on each of those elements also.

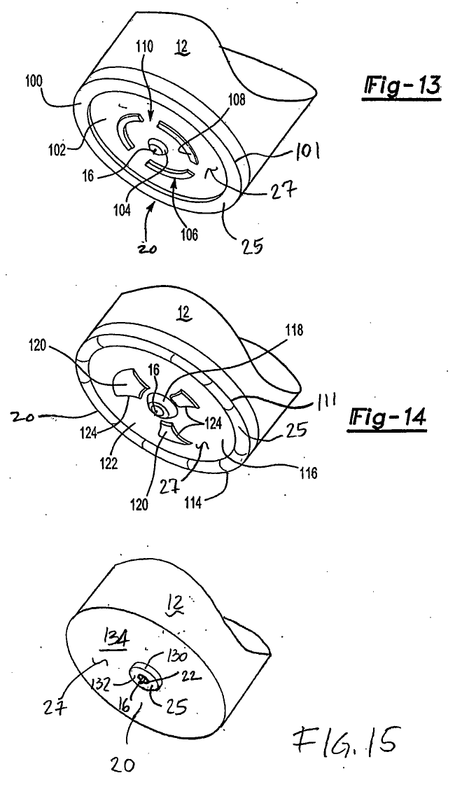

Referring now to FIG. 13, yet another alternative embodiment of the present invention is depicted in which the skin engaging surface 20 is defined by an outer rim 100 that encircles a central surface 102. The central surface 102 is disposed rearward or proximally of the outer rim 100. The outer rim 100 may be either flat or slightly convex. The needle cannula 16 extends outwardly from the skin engaging surface 20 and is immediately surrounded by a protuberance 104 having a blister or bubble like shape. A broken ring 106 may be provided concentrically aligned between the protuberance 104 and the outer rim 100. The broken ring 106 includes a plurality of spaced members 108 each being separated by a space 110. The spaced members 108 extend upwardly from the central surface 102 to a plane generally the same as or slightly below a plane defined by the outer rim 100. The coplanar portions 25 may be defined on the outer rim 100, the protuberance 104, and/or the spaced members 108 depending on the relative heights of the elements. It is preferred that the coplanar portions 25 be defined on the distalmost i portions of the skin engaging surface 20, be it that those portions are defined on the outer rim 100, the protuberance 104, and/or the spaced members 108. The recesses 27 are defined within perimeter 101 of the skin engaging surface 20, and depending on the relative heights of the rim 100, the protuberance 104 and/or the spaced members 108, the recesses 27 may or may not be defined above those elements. During administration of the intradermal injection, the skin of the patient gathers in the recesses 27. Further, the skin may be stretched by the protuberance 104, the spaced members 108, and/or the outer rim 100. If the broken ring 106 is not provided, the skin engaging surface 20 generally has the cross-section shown in FIG. S and discussed above.

An aperture may also be provided through which the needle cannula 16 extends.

Referring now to FIG. 14, still another embodiment of the present invention is depicted in which the skin engaging surface 20 is defined by an outer rim 114 that transitions to a central surface 116. The central surface 116 may be formed with various configurations, such as being flat or convex. The needle cannula 16 extends outwardly away from the skin engaging surface and is immediately surrounded by a protuberance 118 having a blister or bubble like shape.

A plurality of arcuate protuberances 120 encircle the central protuberance 118 and are concentrically aligned between the inner protuberance 118 and the outer rim 114. The outer rim 114 may be flat or convex. A space 122 is defined between each arcuate protuberance 120.

Each arcuate protuberance 120 includes a wall 124 opposing the adjacent protuberance 120.

Each wall 124 preferably defines a convex surface, but may be formed with other configurations such as being flat. The coplanar portions 25 may be defined on the outer rim 114, the protuberance 118, and/or the arcuate protuberances 120 depending on the relative heights of the elements. It is preferred that the coplanar portions 25 be defined on the distalmost portions of the skin engaging surface 20, be it that those portions are defined on the outer rim 114, the protuberance 118 and/or the arcuate protuberances 120. The recesses 27 are defined within perimeter 111 of the skin engaging surface 20, and depending on the relative heights of the rim 114, the protuberances 118 and/or the arcuate protuberances 120, the recesses 27 may or may not be defined above those elements. While administering the intradermal injection, the skin of the patient gathers in the recesses 27. Also, the skin may be stretched by the central protuberance 118, the arcuate protuberances 120, and/or the outer rim 114. An aperture may also be provided through which the needle cannula 16 extends.

In a preferred embodiment, and with reference to FIG. 15, the skin engaging surface 20 includes an annular protrusion 130 which encircles the needle cannula 16. The coplanar portions 25 are defined on a free distal end 132 of the protrusion 130, particularly the distalmost portions of the free distal end 132. It is preferred that the protrusion 130 bound the aperture 22, if used to accommodate the needle cannula 16. The skin engaging surface 20 also includes a secondary surface portion 134 which extends radially from the protrusion 130, above which the recesses 27 are defined. The secondary surface portion 134 may be generally flat, as shown in FIG. 15, or be contoured, e.g., tapered to diverge in a distal to proximal direction. The secondary surface 134 is set back from the free distal end 132.

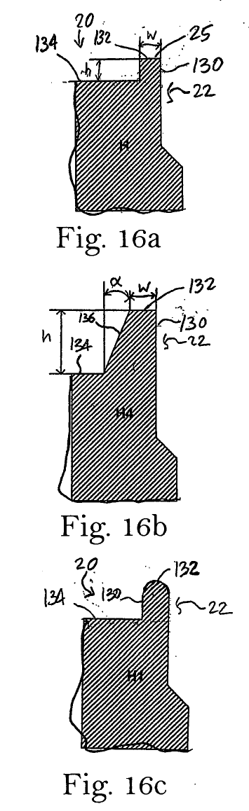

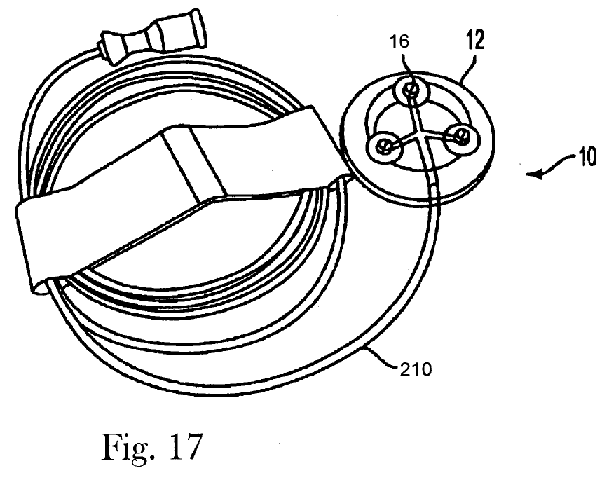

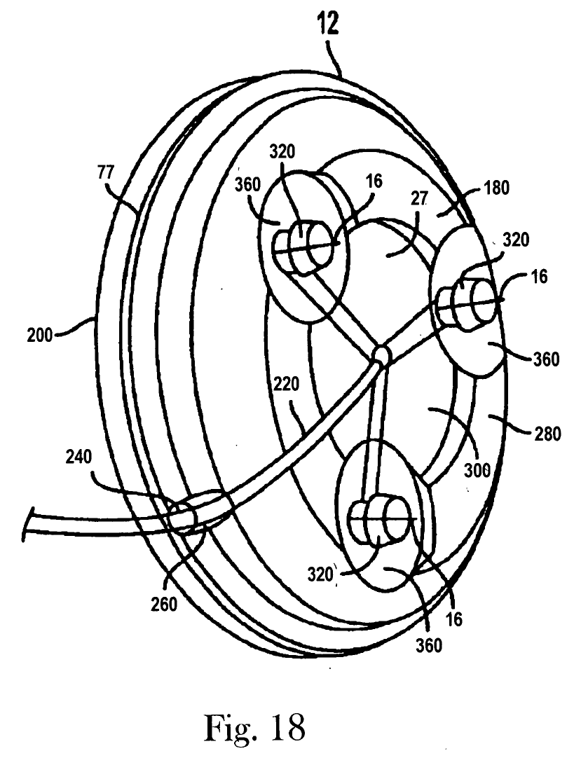

The free distal end 132 may be formed generally planar or with other configurations. As such, the free distal end 132 may define the coplanar portions 25 continuously or discontinuously about the needle cannula 16. In addition, and as shown in FIGS. 16a-16c, the protrusion 130 may be formed with various cross-sections, including rectangular and trapezoidal, although a square cross-section is most preferred. Other polygonal shapes are possible. Also, the protrusion 130 may be at least partially formed arcuately, as shown in FIG. 16c.

With a rectangular cross-section as shown in FIG. 16a, the height h of the protrusion 130 may be in the range of 0.2 mm to 0.5 mm and the width w of the free distal end 132 may be in the range of 0.2 mm to 0.5 mm. Of course, with a square cross-section, the height h and the width w are generally equal. With reference to FIG. 16b, and with a trapezoidal cross- section, the protrusion 130 may have a height h in the range of 0.5 mm to 1.0 mm, a width w of the free distal end 132 in the range of 0.35 mm to 0.6 mm, and a side surface 136 disposed at an angle a, the angle a being in the range of 30-45 degrees.

As indicated above, the various embodiments of the present invention depicted in FIGS. 1-15 are not limited for use with syringes and may be used in connection with any injection device suitable for delivering drug substances to the intradermal region of the skin. As will be appreciated by those skilled in the art, embodiments of the skin engaging surface 20 rely on skin being deformed into at least the recesses 27 defined within the perimeter; of the skin engaging surface 20. The recesses 27 may be in direct communication with the aperture 22 through which the needle cannula 16 passes (e.g., as shown in FIGS. 6 and 11) or may be spaced from the aperture 22 (e.g., with reference to FIG. 8, the annular space between the rims 60 and 62 is spaced from the aperture 22). Beyond the perimeter of the skin engaging i surface 20, a dramatic transition exists to a different oriented surface, such as the cylindrical body of the limiter 12. With the skin engaging surfaces 20 that have gradual transition portions (such as those shown in FIGS. 9 and 10), the gradual transition portions generally face in the same direction as the remaining portions of the skin engaging surface 20. Beyond the perimeters 77 and 82 of the gradual-transition embodiments, a dramatic transition is present to a secondary external surface (e.g., external surface 200 shown in FIGS. 2, 5 and 6) which faces in one or more general directions different from that in which the skin engaging surface 20 faces (e.g., the cylindrical side wall of the limiter 12).

It is preferred that the coplanar portions 25 be located on the distal most portions of the skin engaging surface 20 for the embodiments that include such portions. It is also preferred that the distal tip 34 of the needle cannula 16 be located a distance ranging from 0.5 to 3.0 millimeters from the coplanar portions 25. With reference to FIG. 5, and by way of non- limiting example, distance X from the coplanar portions to the tip of the needle cannula is preferably in the range of 0.5 to 3.0 millimeters.

It is further preferred that skin engaging portions of the skin engaging surface 20 be located about the needle cannula 16 such that an even ring of pressure can be generated about the needle cannula 16 during intradermal injection. Thus, the coplanar portions 25 are preferably located continuously or discontinuously about the needle cannula 16 to contact and provide an even ring of pressure during injection. It is further preferred that the ring of pressure be spaced from the needle cannula 16 to facilitate wheel formation.

Referring now to FIG. 17 and 18, a device 10 having aspects according to the present invention has a body 12 and dermal-access members 16. The device 10 optionally includes tubing 210 for delivering fluid to or removing fluid from the body 12 of the device.

The body 12 optionally has a low profile to lie flat against the skin of a subject. The low profile of the body 12 provides for ease of attachment to the skin and less obstruction to the subject. The low profile can be achieved by reducing the thickness of the body 12. From the previous embodiments and in the embodiment shown, the body 12 has a substantially circular disk shape, although in alternative embodiments, the body 12 can have a non-circular or other more angular shape or be slightly arcuate. As an example, the diameter of the circular body 12 is preferably about 1-10 cm or less, although other sizes and shapes can be used. Embodiments can be manufactured with diameters of 5 mm or smaller.

The body 12, as shown in FIG. 18, has a circular outer side edge 77, a top face 200 and a bottom face 180. The outer side edge 77 preferably has a rounded surface. The rounded surface helps control the pressure distribution on the device 10 and subject during application. Tapering and contouring help tension the skin at a controlled rate to allow the dermal-access members 16 to penetrate the skin with less force than would otherwise be required.

One or more fluid channels 220 are provided in the body 12. The fluid channel 220 has an open inlet end 240. A coupling member 260 is optionally provided for coupling a fluid delivery mechanism to the body 12 at the open inlet end 240. Alternatively, no coupling member is provided and the fluid delivery mechanism is secured directly to the body 12. An axis of the fluid channel 220 optionally extends substantially parallel to the plane of the body 12. In this manner, the body 12 maintains a substantially flat, low profile configuration. Of course, other arrangements of the coupling member 260 and the fluid channel 220 are possible.

In the embodiment shown in FIGS. 17 and 18, the bottom face 180 of the body 12 has first 280 and second 300 surface areas. The first surface area 280 is raised from the body 12 with respect to the second surface area 300. Thus, the second surface area 300 defines a recessed area on the bottom face 180 relative to the first surface area 280.

Raised protrusions 320 are provided in the recessed second surface area 300. As an exemplary embodiment, each protrusion 320 can be formed as a raised conical protrusion. As an alternative, other shapes such as cylindrical shapes may be used. Optionally, a raised conical protrusion 320 can have a flat upper surface to form a conical plateau or lower frustum of a cone. As an alternative, other upper surface shapes and contours may be used.

As shown in FIGS. 17 and 18, the recessed second surface area 300 comprises a central recessed area 27, preferably located in the center of the bottom face 180, and substantially circular recessed areas 360 surrounding each of the protrusions 320. In one embodiment, the recessed second surface area 300, including the central recess 27 and other recesses 360, are recessed at about 1 mm relative to the surrounding first surface area 280, although the depth of the recess can vary from about 0.1 mm and less to about 10 mm. As an example, the recesses 360 surrounding each of the protrusions 320 are about 5 mm in diameter, although the diameter of the recess can vary, for example to about 50 mm. The recesses 360 typically provide an area for the bleb to form. The diameter and arrangement of the recesses 360 and corresponding protrusions 320 can depend on the desired delivery characteristics. Other suitable recess arrangements can be designed depending on the bleb characteristics desired, the volume of substance to be delivered, the rate of delivery of the substance, and other factors. As one option, the diameter of the recess 36 surrounding each of the protrusions 320 can be calculated by one of ordinary skill in the art based on the volume and rate of the fluid administered.

As shown in FIG. 18, the three protrusions 320 and corresponding recessed areas 360 are spaced at 120 degrees relative to one another on the bottom face 180, although arrangements can vary. Some of the alternative arrangement are shown in further embodiments and discussed herein. In the embodiment shown, the center of each protrusion 320 is equally spaced at a distance of about 7.5 mm from the center of the bottom face 180, although, as discussed above, other arrangements can be used depending on the desired delivery characteristics. As an example, the protrusions 320 are about 2 mm in diameter at the top of the protrusion 320 and may have an approximately 10 degrees draft from top to base. The draft of the protrusions 320 can range, for example, from 0 degrees to 60 degrees. The shape and sizes of the protrusions 320 can vary, although typically, the top of the protrusion will range from 0.5 mm or even smaller to about 10 mm in diameter. The diameter and shape of the protrusions 320 can be based on, for example, dermal-access member seating requirements.

In the embodiment shown, one dermal-access member 16 is provided in each conical protrusion 320, although multiple dermal-access members 16 can be provided in each conical protrusion. Thus, in the embodiment shown in FIGS. 17 and 18, three dermal-access members 16 are provided.

The upper surface of the raised conical protrusion may be slightly elevated relative to the first surface area 280, flush with the first surface area 280, or slightly recessed relative to the first surface area 280. It is understood that the relative heights of the respective surfaces may vary depending on desired bleb formation, skin tensioning characteristics, and dermal-access member seating requirements. As an exemplary embodiment, the first surface area 280 will be slightly lower than the top of the protrusions 33, for example 0.25 mm shorter.

Outside of the first surface area 280, the device 10 chamfers to the outer edge 77 to prevent or reduce edge effect, defined as pressure applied to the outer edge of the device that may impede performance of the device 10 or cause the subject discomfort.

In the embodiment shown, each dermal-access member extends about 1 mm from the top of the protrusion 320 with about 0.5 mm to about 2 cm of the dermal-access member remaining within the protrusion 320. In an exemplary embodiment, the device uses hollow dermal-access members 16. The dermal-access member tips can be beveled, for example, at a single bevel angle of approximately 15-35 degrees , preferably 28 degrees .

As shown in FIG. 18, the fluid channel 220 extends between the inlet 240 and the protrusions 320 for supplying a substance to the dermal-access members 16 or for directing a substance withdrawn from a subject to a suitable collection container. In one embodiment, the top face 200 of the body 12 defines the channel 220. Optionally, the channel 220 is open with respect to the top face 200. The channel 220 extends from the opening inlet 240 to each of the dermal-access members 16. In the embodiment shown, the channel 220 includes a central channel 23 from the inlet 240 to the center of the top face 200 and extends from the center outwardly to each protrusion 320.

The device 10 can also include a cover portion (not shown in FIGS. 17 and 18) for covering the channel 220. The cover portion may be glued onto the body 12 with UV cure adhesive or other attachment mechanism.

In the embodiment shown, the tubing 210 delivers fluid to the channel 220. The tubing 210 is secured to the inlet end 240 of the body 12. The tubing 210 may be glued to the coupling member 260. Optionally, the tubing 210 includes 16 gauge catheter tubing with a luer fitting. (not shown) The other end of the tubing can be connected to a supply or receiving device. The supply device may be a syringe (not shown), a unit dose delivery device (not shown), or a suitable metering pump or infusion device (not shown) for delivering a substance to device 10 at a controlled rate. This method can also be used to withdraw a substance from a subject.

In an exemplary embodiment, the channel 220 is smaller than the tubing 210 feeding the channel 220, but significantly larger than the exit diameters of the dermal-access members 16 so as not to result in unnecessary high pressures. The tubing should not be the limiting factor in the flow of substance through the device. Optionally, the size and configuration of the dermal-access member and arrangement of recesses are the primary factors in controlling substance delivery. The body 12 of the delivery device is preferably designed to deliver fluids in the range of about 2-5 psi up to about 200 psi, for example, 50-75 psi. The body 12 can also be designed to deliver at higher and lower pressures. The body and all fitting and components of the device should be rigid enough to withstand pressures on the device without deflection or loss of liquid sealing.

The device 10 may be taped with tape 38, or otherwise secured, onto a subject during application. Alternatively, the device can be manually held in place without any other securing mechanism. The device 10 can also be designed and/or manufactured with tape or other suitable securing mechanism, such as an adhesive, as part of the device 10. Optionally, the device can be installed or incorporated into an applicator device for mechanically applying the device to a user.

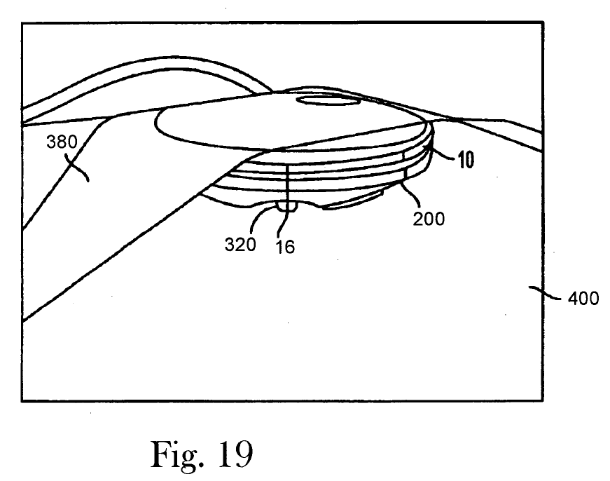

FIG. 19 illustrates the delivery device of FIGS. 17 and 18 in use, taped to the subject 400.

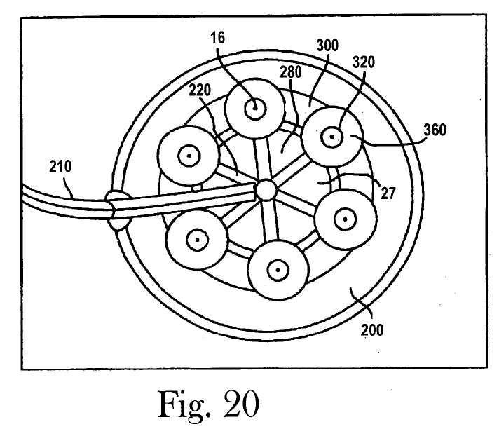

FIG. 20 shows another embodiment of a device having aspects of the invetion. This embodiment is similar to the previous embodiment. However, instead of the three member array shown in FIGS. 17-19, the device shown in FIG. 20 includes a six member array with six protrusions 320 and six dermal-access members 16.

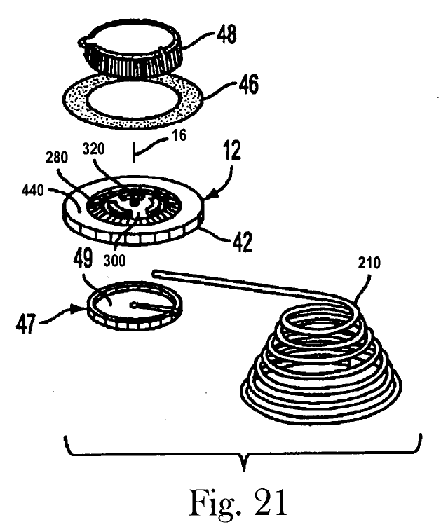

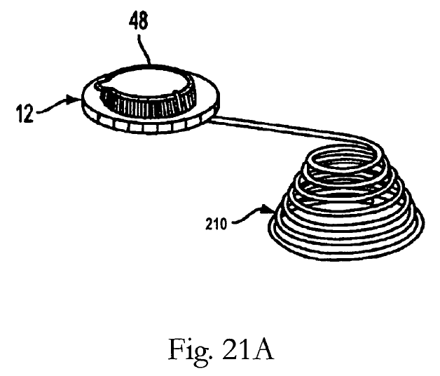

FIG. 21 shows a further embodiment of a device having aspects of the invention. Other than the differences discussed below and illustrated in the Figures, this embodiment is similar to the other embodiments. This embodiment is a single member delivery device 10 with one protrusion 320 and one dermal-access member 16. The device 10 shown in FIG. 21 also differs from the devices of FIGS. 17-20 in that a flange 440 is provided for application of adhesive.

In the example shown in FIG. 21, the body 12 is optionally about 3.8 cm or less in diameter, for example, about 1.2 cm. On the center of the bottom face 180 in the recessed second area 300, the protrusion 320 is formed. In this embodiment, the central recessed area and the circular recessed area are the same area 300 because only one centrally located protrusion 320 is provided. One dermal-access member is installed in the protrusion 320.

A chamfer 42 extends to the edge of the device. The chamfer 42 helps ensure that the proper pressure is applied to the dermal-access member 16 and prevents any adverse effect of the edge from the device during delivery.

In the embodiment shown, the flange 440 surrounds the edge 45 for application of an adhesive ring 46. The flange 440 can, for example, extend about 1 cm beyond the edge of the device. The flange can be rigid or flexible and can be designed to extend as far as necessary beyond the edge of the body 12, depending on the necessary level of securement and its placement on the subject. The flange 440 should be slightly recessed relative to the first areas 280 to compensate for the thickness of the adhesive 46, and to minimize or eliminate interference with the delivery area. For example, the flange can be recessed 1 mm although the amount the flange 440 is recessed can vary. Generally, the adhesive 46 should be located at a distance from the delivery site, preferably, as far away as is practical, so as not to interfere with delivery characteristics.

The adhesive 46 is preferably a pressure sensitive adhesive capable of attaching the device 10 to the surface of the skin of a subject and is preferably applied directly to the flange 440. The adhesive 46 can be a double-faced adhesive foam tape having one face bonded to the flange 440. The device 10 is preferably packaged with a release sheet covering the adhesive 46 that can be removed immediately before use. As an alternative, any suitable means for maintaining biological interface of the device with a subject may be used. The flange 440 and adhesion arrangement 46 can also be provided in the other embodiments.

The top face 200 of the body 12 defines a channel 220 for insertion of tubing 210 for delivery of the fluid. This feature may be present in the other embodiments, although not clearly shown in previous figures. The channel 220 may extend from the edge of the main body 12 to the center of the top face 200 of the body 12 and is in fluid communication with the dermal-access member 16. In the exemplary embodiment, the tubing extends into the body to a narrowing stop in the channel. However, the device can be designed with the tubing extending only to the edge of the device or all the way through the channel to the dermal-access members. The channel 220 can be, for example, about 1 mm in diameter, although the channel can be modified depending on the desired delivery characteristics, including delivery rate and volume. The channel 220 can narrow as necessary to reduce any dead space inside the device but outside the tubing. For example, the channel can be, for example, 0.5 mm in diameter or less. Dead space results in wasted substance remaining in the device and not delivered to the subject and/or requires more pressure than would otherwise be necessary to deliver the substance to the subject. The top face 200 of the body 12 also has a raised area on the center of the top face 200. The raised area has a wall or rib surrounding the fluid channel 220 to enhance sealing of the channel 220 and to prevent any adhesive from wicking into the fluid channel during assembly. As an example, the rib can be about 0.5 mm in height.

A cover portion 47 is provided to seal the fluid channel 220. The cover portion 47 has an inside face and an outside face (not shown). Preferably, the cover portion 47 is circular with a recess 49 on the inside face that accommodates the raised area (not shown in FIGS. 21 and 21A) on the top face 200 of the body 12. As an example, the cover portion 47 can have a diameter corresponding to the body 12 of the device 10. The recess 49 can be deep enough to accommodate the corresponding raised area of the body. The recess 49 and raised area of the body act as a locating aid for placement of the cover portion. The inside of the cover portion 47 can also define a groove (not shown) which mates with the rib on the top face 200 of the body 12. Preferably, the groove is more shallow than the rib to prevent any possible wicking of adhesive. The rib on the top face 200 allows for location and alignment of the cover portion 47. The cover portion 47 and raised area can also be designed to account for adhesive used to adhere the cover portion to the body 12. The cover portion 47 defines a mating half of the fluid channel 220 to allow for obstruction free insertion of the tubing 24. The cover portion 47 can be of sufficient thickness to help reduce deflection of the cover portion when pressurized. As an option, the cover portion 47 should not be set on the flange 440, but instead, on the body, which, as discussed above, is of a rigid design to prevent deflection.

Shield 48 can be provided for protecting the dermal-access member 16 before use. As shown in FIG. 21, the shield 48 can have a tabbed lid with three slots to allow it to be press fitted inside the diameter of the adhesive ring. Alternatively, the shield 48 can have any suitable design which protects the dermal-access member prior to use. FIG. 21A shows the assembled device from FIG. 21.

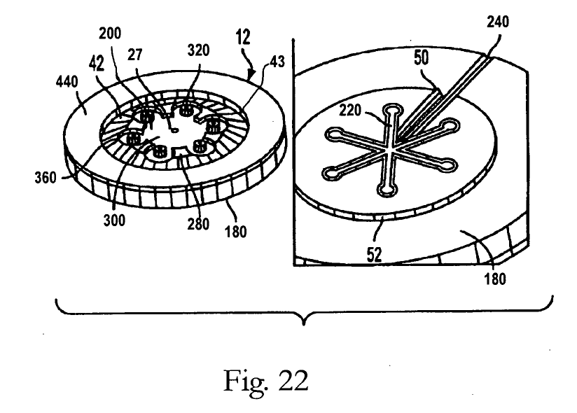

FIG. 22 shows another embodiment having aspects of the invention. This embodiment is similar to the embodiment shown in FIG. 21. The bottom face 180 of the body has a six member array of six protrusions 320 and six dermal-access members 16. The bottom face 180 has a raised first surface area 280 and a recessed second surface area 300. The protrusions 320 are provided on the second surface area 300. The bottom face 180 also has a chamfered surface 42 extending from the first surface 28 to the edge 43. A flange 440 is provided for application of adhesive. FIG. 22 also shows the top face 200 of the body 12. Fluid channel 220 is shown extending from the inlet port 24 at the edge of the body to the center of the body 12. The fluid channel 220 also extends from the center of the device to each protrusion 320 to deliver fluid to the dermal-access members 16. A cover portion (not shown) can be provided to enclose the open channel.

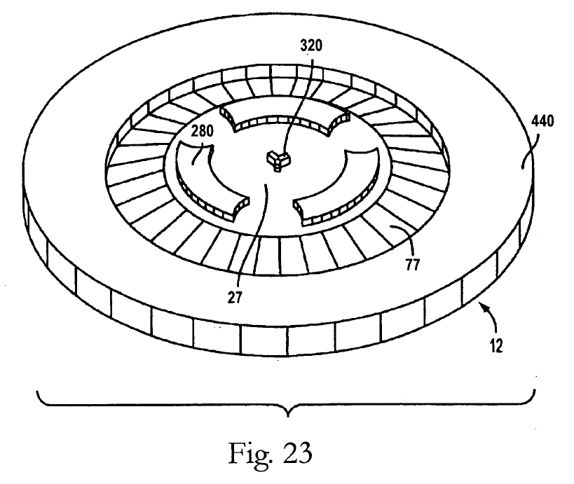

FIG. 23 is an enlarged perspective view of the bottom face of another embodiment having aspects of the invention. The bottom face 180 of the body 12 shown in the embodiment of FIG. 23 is similar to the device shown in FIG. 21. The embodiment of FIG. 23 is a single member array with a single protrusion 320. Instead of being a conical protrusion, the protrusion 320 has arms extending at 120 degrees from one another. The device of FIG. 23 has a three portion first surface area 280 and an edge 16 that chamfers to the flange 440.

As shown by the alternate protrusion shown in FIG. 23, the protrusions of any of the embodiments can be any suitable shape or arrangement to achieve optimal results. For example, the protrusions can have cylindrical, pyramidal, or other geometrical configurations. As a further alternative, the protrusions can be arranged as a type of sleeve supporting the dermal-access member which retracts upon application. The protrusions can be arranged on a flexible hinge region, such as a flexible membrane or temperature sensitive polymer, which also retracts in a longitudinal direction upon application. In addition, the upper surface of the protrusion can be flat, concave or convex. Alternatively, the dermal-access member can be supported directly on the second surface area without any protrusion or with a protrusion that provides minimal support.

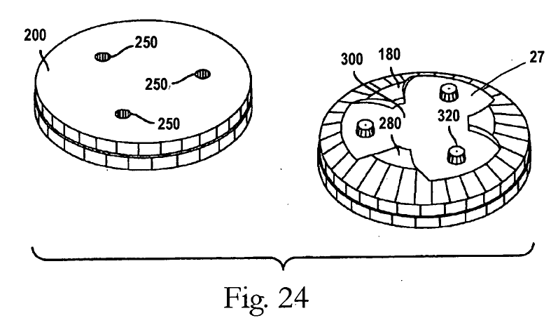

FIG. 24 is a perspective view of the top 200 and botton 180 faces of another embodiment having aspects of the present invention. The device shown in FIG. 24 is a three member array with three protrusions. Instead of having a longitudinal channel defined on the top face of the body, which extends from the edge of the device to a dermal-access member, the embodiment of FIG. 24 has individual channels 250 in fluid communication with the dermal-access members (not shown in FIG. 24). In the embodiment shown, the individual channels 250 extend perpendicularly directly from the top face 200 to the protrusions 320 and the dermal-access members. Any suitable mechanism, such as a syringe or pump, can be used to deliver or extract fluid from the individual channels 250. Individual channels 220 can be useful in delivering different fluids to a subject or delivering fluids at different pressures. For example, as shown in FIG. 24, three separate delivery means could deliver fluid to the device.

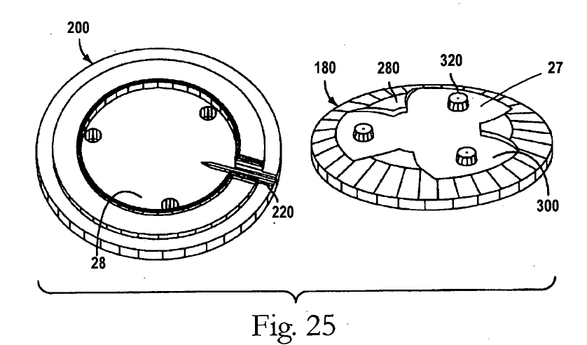

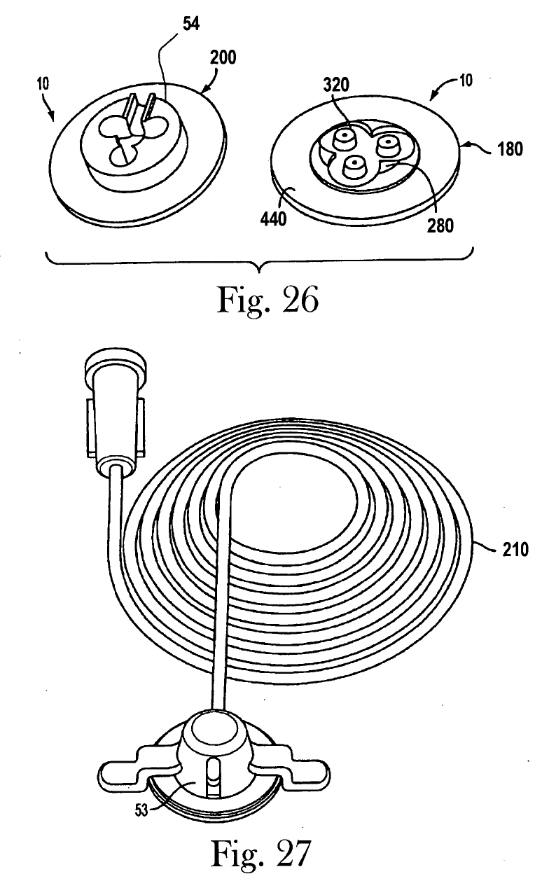

FIG. 25 is a perspective view of the top 200 and bottom faces 180 of another embodiment having aspects of the present invention. The device shown in FIG. 25 is a three point array with three protrusions 320. Instead of having a longitudinal channel defined on the top face of the body which extends from the edge of the device to a dermal-access member, the embodiment of FIG. 25 has a reservoir 23 defined on the top face 200. Fluid is introduced from the relatively shorter longitudinal channel into the reservoir 28. The fluid is communicated from the reservoir 28 to the dermal-access member (not shown in FIG. 25).

FIGS. 26-29 show still further embodiments of a device having aspects of the invention. Generally, the embodiments shown in FIGS. 26-29 are smaller than those shown in FIGS. 17-19 and 20-25.

The device 10 shown in FIGS. 26 and 27 is a three member array with a bottom face 180 having three protrusions 320 and a flange 440. As shown in FIG. 26, the dermal-access members have not yet been installed. The top face 200 has a raised portion 54 at least in part defining flow paths to the protrusions and configured to receive a cap assembly 53. The cap assembly 53 and tubing 210 for delivering the fluid to the patient during use is shown in FIG. 27.

As an example, the device 10 shown in FIGS. 26 and 27 has a thickness of about 5 mm and a diameter of about 18 mm with the flange 440. The body chamfers at 45 degrees to the flange 440. The protrusions 320 extend slightly above the raised first surface area 280, for example about 0.2-0.3 mm above the first surface area 280. The top face of each of the protrusions 320 is about 2 mm in diameter. The protrusions 320 are spaced equally around the center of the top face 20, and the distance from the center of a protrusion 320 to the center of the device 10 is 2.5 mm.

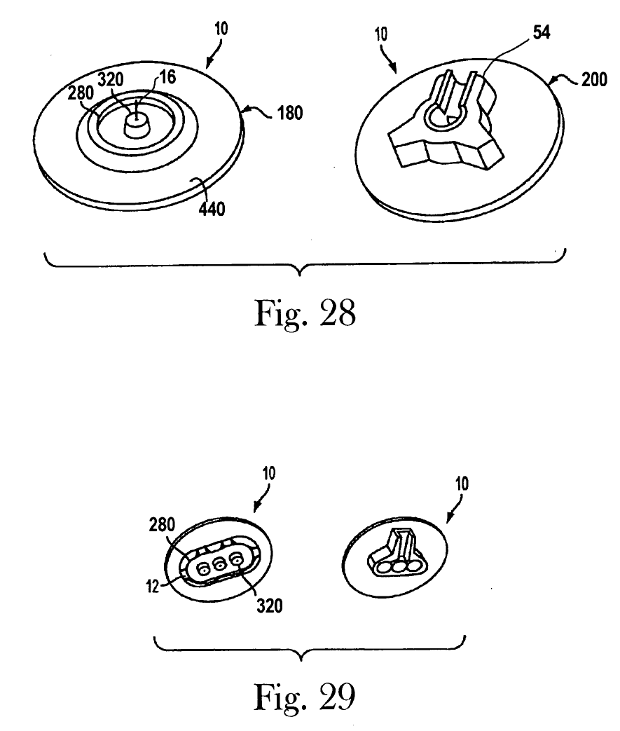

The device 10 shown in FIG. 28 is a single dermal-access member device with a bottom face 180 having a single dermal-access member installed in the protrusion 320. The top face 200 has a raised portion 54 at least in part defining a flow path to the protrusion and configured to receive a cap assembly (not shown).

By way of example, the device 10 shown in FIG. 28 is about 5 mm thick and has a diameter of about 18 mm with the flange 440. The protrusion 320 extends slightly above the raised first surface area 280, for example about 0.2-0.3 mm above the first surface area 280. The top face of the protrusion 320 is about 2 mm in diameter.

The device 10 shown in FIG. 29 is a three dermal-access member linear array with a bottom face 180 having three protrusions 320. The top face 200 has a raised portion 54 at least in part defining flow paths to the protrusions and configured to receive cap assembly (not shown). The dermal-access members are not yet installed in FIG. 29. Both the device 10 and body 12 are elliptical.

By way of example, the elliptical embodiment of the device 10 shown in FIG. 29 is about 5 mm thick and has length of about 19.5 mm and a width of about 23 mm. The body 12 has a length of about 15 mm and a width of about 9 mm. The protrusions 320 extend slightly above the raised first surface area 280, for example about 0.2-0.3 mm above the first surface area 280. The top faces of the protrusions 320 are about 2 mm in diameter, and the center of a protrusion is spaced about 3 mm from an adjacent protrusion.

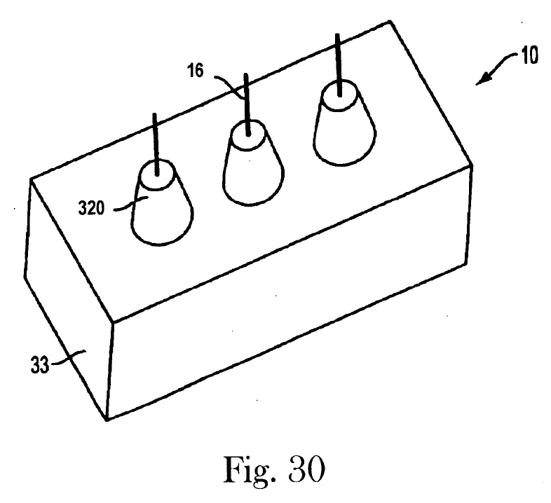

Another embodiment of the dermal-access member array is shown in FIG. 30. It includes a linear dermal-access member array with a manifoled 33 for holding the protrusions 320 and dermal-access members 16 having a rectangular face and a generally parallelpiped shape. Typically, the embodiment shown in FIG. 30 is integrated into device 10.Other than the protrusions, the embodiment of FIG. 16 has a planar face. The face can have a length of about 4.8 mm, and a width of about 11 mm. The protrusions have a linear arrangement and are spaced about 3 mm apart from one another. The diameter of the conical protrusions are relatively small, for example, about 0.95 mm or smaller.

The arrangement and relative heights of the dermal-access members, recesses, and protrusions can be modified to accomplish or emphasize any number of intended beneficial characteristics of the invention. Specifically, the length, width and spacing of the dermal-access members can vary depending on the pharmaceutical agent being administered or required to penetrate the skin to the optimum depth for the specific pharmaceutical or bioactive agent being administered. The device of the present invention maximizes the effective penetration of dermal-access members to a targeted depth. The device can control the size of the bleb. In a device with multiple dermal-access members, the device can be engineered to control the instillation patterning of individual blebs and their relationship to each other. Non-communication between individual dermal-access members can be meaningful for deposition of large volumes in a broad biological space or the deposition of multiple fluids, or in designing the pressure parameter of a dermal-access member. The device can be designed to provide sufficient fluid flow path to accommodate the desired velocity and rate of fluid to be instilled and to minimize the amount of void volume. The device can further be designed as a function of the desired bleb pattern and for application of a particular fluid at a particular site to minimize the area of application.

Generally, the patterning of the dermal-access members can be designed to achieve desired characteristics. Typically, a minimal number of dermal-access members can be used to reduce the pain or the perception of pain by a subject, manufacturing complexity or cost, the number of potential failure points, the complexity of the device fluid dynamics, and the dose lost to void volumes in the device or system. The number of dernal-access members can be increased to decrease the possibility of blocked fluid paths, to increase the distribution area of instilled fluid to accommodate a greater volume or delivery rate, and to potentially increase uptake.

Alternate arrangements for delivering fluid to the dermal-access members include but are not limited to multiple reservoirs; a manifold arrangement in which fluid is communicated from a reservoir, through individual channels to the dermal-access members; and independent channels. In addition, the channels can be provided with individual or combination valving or other means for fluid flow rate control.

As discussed above, the number and arrangement of dermal-access members and protrusions in each of the embodiments can depend on the desired range of fluid delivery volume. Furthermore, the recessed second surface area surrounding each protrusion can be arranged based on the desired range of fluid delivery volume. For example, a three member array that delivers 100 μl of fluid may have recesses surrounding each dermal-access member of approximately 5 mm in diameter. Conversely, a single member array that delivers 100 μl of fluid may have a recess surrounding the single dermal-access member with an approximately 10 mm diameter. As discussed above, the size and arrangement of the recesses depend on the desired flow characteristics, including the volume and rate of delivery of the substance.

A method for delivering or withdrawing a substance through the skin is also provided. The device is positioned in a target site on the surface of a subject's skin. The body is pressed downwardly against skin with a pressure sufficient to cause dermal-access members to penetrate the layers of skin. The depth of penetration is dependent upon the length of dermal-access members, the spacing of the dermal-access members, and the dimensions of the body, including the height of the protrusion, pressure exerted on the device, and the tensioning of the skin resulting from the body.

The skin of a subject has elastic properties that resist penetration by the dermal-access members. The skin can be stretched by the raised first surface area until the skin is taut before the dermal-access members penetrate the skin. A penetrating pressure can be applied to the device until the first surface area contacts the skin. This promotes uniform penetration of the skin by each of the dermal-access members. Consequently, when the device is secured to skin with either a manual application or adhesive, a pressure is constantly applied to dermal-access members 16.

A substance is supplied to the device and fed to dermal-access members for delivery to the subject. In alternative embodiments, a substance is withdrawn from the subject in a similar manner.

For a bolus type injection, the spacing of the delivery points is not as important because the pressure is higher and delivery occurs at each dermal-access member approximately simultaneously. Dermal-access member spacing in the bolus type injection may determine whether a single bleb or multiple blebs form.

For lower rate deliveries, it is beneficial to ensure that the delivery points are spaced close enough together to create a single bleb. As delivery at a particular dermal-access member in a multi-dermal-access member device begins, the pressure at that particular dermal-access member decreases. At relatively low delivery pressures, if the dermal-access members are spaced too far apart, the first dermal-access member to form a bleb will be the preferential path because the substance to be delivered will inherently follow the path of least resistance. Thus, by having all the points feed the same bleb, no preferential flow through a particular dermal-access member or delivery point should occur because pressure will be equalized across the dermal-access members.

A device having aspects of the invention can remain interfaced with the skin for sufficient time to withdraw from or deliver to the subject the desired substances. The length of time the device is required to be attached or in communication with the skin of the subject is usually dependent on the substance being delivered or withdrawn, the volume of the substance, the target area on the skin, the depth of penetration, and the number and spacing of dermal-access members. The amount of time the device is secured to the skin may reduce the amount of leakage from the skin after delivery of the fluid.

Many of the considerations in designing the device of the present invention involve proper placement of the dermal-access members, including placement of the dermal-access members at the proper depth. Specifically, pharmacokinetics (PK) for certain classes of medicaments can be improved by administering the medicament at a specified place below the stratum corneum.

Generally, deposition in intradermal tissue results in faster drug onset kinetics for system uptake and bioavailability, and increased bioavailability for some drugs. However, intradermal delivery is limited in that intradermal tissue space is highly compact and has limitations on the total amount of volume which can be administered, the rate at which such fluid can be administered, and the pressure required to administer such volume. Generally, the subcutaneous layer is not well perfused by capillaries. As such, absorption is both slower, and in some cases, decreased bioavailability.

Thus, the PK outcome of dermal-access delivery is specific to the deposition depth and patterning of the administered fluid and such deposition can be mechanically controlled via design of the device of the present invention. It has been shown that delivery of medicaments to two different depths increases the PK benefits, for example, delivery to both shallow subcutaneous areas and intradermal areas.

The present invention can include a device to deliver the medicament to two different depths, and specifically, to two different physiological tissue compartments, such as shallow subcutaneous and intradermal. This can be accomplished, for example, by dermal-access members of different lengths. Other geometric or mechanical mechanisms can also be designed to deliver fluids to different depths. The device can also be provided with flow restrictors to deliver differing amounts of fluid to different areas.

For each of the embodiments discussed herein, the device is optionally radiation stable to allow for sterilization, if radiation is to be used. Optionally, the body should be transparent or translucent to allow for light to penetrate and cure the UV adhesive holding the dermal-access member secure. As another option, the body can be opaque and epoxy can be used to secure the dermal-access member. It is noted that having a transparent body enables a user or other person administrating the device to properly prime the device by ensuring that no excess air is in the device. Furthermore, the body and cover portion material should be stiff enough so as not to deflect during normal use conditions and should be able to withstand internal fluid pressure in the range of about 2-5 psi to about 200 psi without failure or leaks. However, the flange and adhesive can be as flexible as necessary for comfortable and secure attachment to the subject. The body and cover portion material can selected to be non-affected by the drug and having no effect on the drug candidates to be used. The body and the cover portion material should also be hypoallergenic.

The device of the invention can optionally be used as a disposable, single-use device. The device can be sterilized and can be stored in a suitable sterile package.

Adequate dermal-access member seating is an important aspect of the present invention. Successful dermal-access member seating is defined as positioning the dermal-access members in the skin such that fluid delivered through the dermal-access member, or dermal-access members does not leak out of the skin.

Generally, there are four factors which contribute to a desirable dermal-access member seating: dermal-access member length, dermal-access member protrusion geometry, dermal-access member overtravel, and the dermal-access member seating velocity. Overtravel is defined as the extent that the upper face of the protrusion extends beyond the adhesive or other securing mechanism of the device i.e., the bottommost face of the device. The embodiment shown in FIG. 26 has an overtravel of about 1 mm, although more or less overtravel amounts can be adequate to ensure proper dermal-access member seating, for example, about 0.5 mm. Of course, it is also important to avoid any obstructions on the body face.

Exemplary embodiments of the geometry of the device in general and of dermal-access member manifolds have been discussed above.

Experiments have shown that smaller protrusion diameters increase the effectiveness of dermal-access member seating. It was believed that the higher local pressure exerted by the smaller surface of the protrusion for a given force contributes to the beneficial dermal-access member seating. It is further believed that the smaller surface area of the face of the protrusion has a smaller local effect on the development of the bleb.

In one such experiment, a device was applied to a swine test subject to determine the effectiveness of smaller diameter protrusions as compared to larger diameter protrusions. The experiment was conducted at a constant delivery pressure of 15 psi, with a 50 μL air bolus, and with needles as the dermal-access members. The protrusions are conical protrusions with a flat top surface. The dermal-access members extend 1 mm above the top surface of the protrusion. Although the surface is flat in this experiment, as noted above, the top surface of the protrusion can be concave or convex. If the top surface is concave, the length of the dermal-access member is measured from the outer rim of the top surface to the top of the dermal-access member. If the top surface is convex, the length of the dermal-access member is measured from the uppermost tangent of the surface to the top of the dermal-access member.

In the aforementioned experiment, the smaller diameter protrusions are about 1 mm (0.0375″) in diameter and the larger diameter protrusions are about 2 mm (0.075″) in diameter. The experiment also accounted for varying amounts of overtravel. The results are shown in Table 1. Column “over” describes the amount of overtravel in thousandths of an inch. Column “leaker” states whether the trial leaked or not. Column “bleb type” describes the number and particulars, if any. Column “average rate” describes the average steady-state flow rate calculated in μL/min. The average rate of a trial that leaked is 0. Column “if no leaks” shows the average rate of the properly seated trials.

TABLE 1
CONSTANT PRESSURE: 15 PSI; 50 uL AIR BOLUS; 1 mm NEEDLES
SMALL 0.0375″ d CONE GEOMETRY

| | | | | 144 min. | | |
|---|---|---|---|---|---|---|
| CONE | OVER | EXP | LEAKER | BLEB TYPE | AVG RATE | IF NO LEAKS |
| SMALL | 0 | — | Y | | 0 | |
| SMALL | 0 | 10 | N | 3 | 61.1 | 61.1 |
| SMALL | 0 | — | Y | | 0 | |
| SMALL | 0 | 15 | N | 3 | 46.6 | 46.6 |
| SMALL | 0 | — | Y | | 0 | |
| SMALL | 0 | 29 | N | 3 | 29.1 | 29.1 |
| SMALL | 0 | — | Y | | 0 | |
| | | | | AVERAGE | 19.54 | 45.60 |
| | | | | STDEV | 26.07 | 16.02 |
| SMALL | 20 | 2 | N | 3 | 12.6 | 12.6 |
| SMALL | 20 | 7 | N | 3 | 46.9 | 46.9 |
| SMALL | 20 | 11 | N | 3 | 62.4 | 62.4 |
| SMALL | 20 | 16 | N | 2 | 12.7 | 12.7 |
| SMALL | 20 | 20 | N | 3 | 66.8 | 66.8 |
| SMALL | 20 | 21 | N | 3 | 8.4 | 8.4 |
| SMALL | 20 | 32 | N | 3 | 31.7 | 31.7 |
| SMALL | 20 | 37 | N | 3 | 22.8 | 22.8 |
| | | | | AVERAGE | 33.04 | 33.04 |
| | | | | STDEV | 23.11 | 23.11 |
| SMALL | 40 | 5 | N | 3 | 173.5 | 173.5 |
| SMALL | 40 | 12 | N | 3 | 30.7 | 30.7 |
| SMALL | 40 | 19 | N | 3 | 22.8 | 22.8 |
| SMALL | 40 | 24 | N | 3 | 54.3 | 54.3 |
| SMALL | 40 | 30 | N | 18IG, 2SM | 29.6 | 29.6 |
| SMALL | 40 | 33 | N | 3 | 18.7 | 18.7 |
| | | | | AVERAGE | 54.93 | 54.93 |
| | | | | STDEV | 59.39 | 59.39 |
| REG | 0 | — | Y | | 0 | |
| REG | 0 | — | Y | | 0 | |
| REG | 0 | 17 | N | 3 | 67.5 | 67.5 |
| REG | 0 | 23 | N | 3 | 44.2 | 44.2 |
| REG | 0 | — | Y | | 0 | |
| REG | 0 | — | Y | | 0 | |
| REG | 0 | — | Y | | 0 | |
| | | | | AVERAGE | 15.96 | 55.85 |
| | | | | STDEV | 28.07 | |
| REG | 20 | — | Y | | 0 | |
| REG | 20 | 18 | N | 3 | 42.4 | 42.4 |
| REG | 20 | 25 | N | 3 | 110.8 | 110.8 |
| REG | 20 | 31 | N | 3 | 27.2 | 27.2 |
| REG | 20 | 38 | N | 3 | 50.1 | 50.1 |
| | | | | AVERAGE | 46.10 | 57.63 |
| | | | | STDEV | 40.92 | 36.70 |
| REG | 40 | 4 | N | 3 | 31.7 | 31.7 |
| REG | 40 | — | Y | | 0 | |
| REG | 40 | 13 | N | 3 | 156.2 | 156.2 |
| REG | 40 | 28 | N | 2, 1BL NEED | 16.5 | 16.5 |
| REG | 40 | 34 | N | 3 | 32.3 | 32.3 |
| | | | | AVERAGE | 47.34 | 59.18 |
| | | | | STDEV | 62.28 | 65.10 |

As can be seen from Table 1, the smaller diameter protrusions provided better needle seating. In addition, overtravel was shown to be a factor in needle seating. The experiment suggested that overtravel greatly prevents leaking.

Interestingly, overtravel did not seem to negatively affect infusion rates. This was somewhat surprising, given the previous experience with overdriven or overtraveled needles. It has been the conventional experience when using 1 mm needles mounted in catheter tubing that pushing the catheter into the skin significantly affects the pressure required to infuse at a given rate in a constant pressure system. However, the amount of overtravel necessary to produce this effect is likely larger than the maximum overtravel of 0.040″ seen in this experiment. This suggests an optimal overtravel amount which can be discerned from further experiments.

It has further been shown that an increased velocity in the application of the dermal-access members can increase the effectiveness of the seating.

An applicator for mechanically applying the device to a patient can control the velocity of the dermal-access members. For example, an applicator such as a Minimed SOF-SERTER™ insertion device or a BD INJECT-EASE™ device can be modified to apply the device to a user at a desired velocity. The device is driven toward the skin by springs contained in the applicator and results in the dermal-access members seating into the skin of a subject. Among other factors, the strength of the springs determines the velocity of the dermal-access members.

Experiments have shown that there is a continuum of velocity ranges within which dermal-access member seating improves with velocity, for a given skin type, manifold mass, and needle sharpness.

Initial seating experiments in Yorkshire pigs utilized a single spring rate of about 5 lbf/in. This allowed a 1.7 gram manifold to be propelled at about 6.3 m/s. At this velocity, most 1 mm and 3 mm dermal-access members seated without leaking. However, a large number of manifolds did not have enough energy to seat the dermal-access members to the required depth. Heavier manifold tests, from a drop-center design, had velocities of about 3 m/s. At this velocity, most of the 1 mm dermal-access members leaked. Similarly, most of the 3 mm dermal-access members produced very shallow blebs. One manifold arrangement uses two springs with spring constants of 3.2 lb/in, and is less massive than other manifolds. This manifold arrangement enables a manifold velocity of about 12 m/s or greater. With this arrangement, nearly 100% of the dermal-access members seated properly. Accordingly, it has been shown that, for this arrangement, a velocity of about 6 m/s to 18 m/s is ideal, optionally about 6 m/s to about 25 m/s. It is noted, however, that these resultant, calculated velocities were calculated based on energy conservation equations based on known initial forces, and does not account for any friction within the applicator or friction of the dermal-access members passing through the skin. The actual velocities in this example could be much less, for example, 50% less.

One experiment determining dermal-access member velocity utilizes a mechanical applicator in which a device with a three dermal-access member manifold is loaded. In this experiment, 34 gauge dermal-access members are used. A coil spring is placed on a post of the manifold to tension the manifold in the applicator. A luer and line arrangement can supply fluid to the manifold at a constant pressure. The applicator is placed on a swine, the applicator is activated to release the spring to drive the manifold with the dermal-access members into the skin, and fluid is delivered to the subject. In this experiment, the manifold is driven about 5 mm. The following parameters were considered:

Springs Force: None;

Low: 1 lb. initial spring force, 0.5 lb. final force; or

High 2 lb. initial spring force, 1 lb. final force

Device: Center or Side

Adhesive: Full or Missing (safety)

Septum: With or Without

Member Length: 1 mm or 3 mm

The results are shown in TABLE 2. As can be seen, needle seating increases with velocity.

TABLE 2
SUMMARY SHEET: DOINK2
CONSTANT PRESSURE: 15 PSI; AIR BOLUS

| | | | SPRING | | | | RATE | |
|---|---|---|---|---|---|---|---|---|
| EXP # | NEEDLE | DEVICE | FORCE | SEPTUM | SAFETY | BLEBS | (uL/min) | LEAKER |
| 27 | 1 | C | NONE | N | N | | 0 | Y |
| 26 | 1 | C | NONE | N | N | | 0 | Y |
| 23 | 1 | C | LOW | N | N | 2 | N/A | Y |
| 24 | 1 | C | LOW | N | N | 1 | N/A | N |
| 70 | 1 | C | LOW | N | N | 3 | 44.1 | N |
| 97 | 1 | C | LOW | N | N | | 0 | Y |
| 49 | 1 | C | LOW | N | Y | ID | 72.6 | N |
| 51 | 1 | C | LOW | N | Y | | 0 | Y |
| 25 | 1 | C | LOW | Y | N | | 28.6 | N |
| 26 | 1 | C | LOW | Y | N | | 33.18 | N |
| 62 | 1 | C | LOW | Y | N | 3 | 70 | N |
| 64 | 1 | C | LOW | Y | N | 1 | 0 | Y |
| 53 | 1 | C | LOW | Y | Y | | 0 | Y |
| 55 | 1 | C | LOW | Y | Y | | 0 | Y |
| 100 | 1 | C | LOW | Y | Y | 3 ID | 76 | N |
| 102 | 1 | C | HIGH | N | N | 2 | 0 | Y |
| 104 | 1 | C | HIGH | N | N | | 0 | Y |
| 105 | 1 | C | HIGH | N | N | 2 | 0 | Y |
| 50 | 1 | C | HIGH | N | Y | ID | 117.6 | N |
| 52 | 1 | C | HIGH | N | Y | | 103.4 | N |
| 65 | 1 | C | HIGH | N | Y | 3 | 159.3 | N |
| 105 | 1 | C | HIGH | Y | N | | 0 | Y |
| 107 | 1 | C | HIGH | Y | N | | 32.9 | N |
| 106 | 1 | C | HIGH | Y | N | 1 | 0 | Y |
| 54 | 1 | C | HIGH | Y | Y | 3 | 36.8 | N |
| 56 | 1 | C | HIGH | Y | Y | 3 | 98.4 | N |
| 58 | 1 | C | HIGH | Y | Y | | 0 | Y |
| 7 | 3 | C | LOW | N | N | | 628.1 | N |
| 8 | 3 | C | LOW | N | N | | 563.4 | N |
| 74 | 3 | C | LOW | N | N | | 633.5 | N |
| 41 | 3 | C | LOW | N | Y | 1 ID | 6.72 | N |
| 43 | 3 | C | LOW | N | Y | 3 | 114.8 | N |
| 56 | 3 | C | LOW | N | Y | | 475.4 | N |
| 9 | 3 | C | LOW | Y | N | | 664.8 | N |
| 10 | 3 | C | LOW | Y | N | | 679.2 | N |
| 51 | 3 | C | LOW | Y | N | | 685.8 | N |
| 45 | 3 | C | LOW | Y | Y | ID | 19.7 | N |
| 47 | 3 | C | LOW | Y | Y | ID | 34.5 | N |
| 80 | 3 | C | LOW | Y | Y | 3 | 1172.6 | N |
| 83 | 3 | C | LOW | Y | Y | | 447.8 | N |
| 67 | 3 | C | HIGH | N | N | 3 | 386.2 | N |
| 69 | 3 | C | HIGH | N | N | | 870.9 | N |
| 89 | 3 | C | HIGH | N | N | | 570.3 | N |
| 42 | 3 | C | HIGH | N | Y | SC | 486.5 | N |
| 44 | 3 | C | HIGH | N | Y | 1 | 20.3 | N |
| 73 | 3 | C | HIGH | N | Y | 3 | 1080 | N |
| 71 | 3 | C | HIGH | Y | N | 3 | 1300.3 | N |
| 72 | 3 | C | HIGH | Y | N | 3 | 754.2 | N |
| 82 | 3 | C | HIGH | Y | N | | 61.9 | N |
| 46 | 3 | C | HIGH | Y | Y | | 503.2 | N |
| 48 | 3 | C | HIGH | Y | Y | | 543.6 | N |
| 77 | 3 | C | HIGH | Y | Y | | 141.2 | N |
| 11 | 3 | C | NONE | N | N | | 1008.5 | N |
| 12 | 3 | C | NONE | N | N | | 358 | N |
| 19 | 1 | S | NONE | N | N | | 1841.4 | N |
| 20 | 1 | S | NONE | N | N | | 0 | Y |
| 17 | 1 | S | LOW | N | N | | 44.1 | N |
| 10 | 1 | S | LOW | N | N | | 0 | Y |
| 76 | 1 | S | LOW | N | N | 3 | 73.2 | N |
| 29 | 1 | S | LOW | N | Y | 3 | 88.7 | N |
| 30 | 1 | S | LOW | N | Y | 3 | 119.6 | N |
| 60 | 1 | S | LOW | N | Y | | 0 | Y |
| 21 | 1 | S | LOW | Y | N | | N/A | N |
| 22 | 1 | S | LOW | Y | N | | 0 | Y |
| 31 | 1 | S | LOW | Y | N | | 0 | Y |
| 32 | 1 | S | LOW | Y | N | 3 | 188.5 | N |
| 59 | 1 | S | LOW | Y | Y | | 0 | Y |
| 61 | 1 | S | LOW | Y | Y | 3 | 56.1 | N |
| 91 | 1 | S | LOW | Y | Y | 1 ID | 0 | Y |
| 92 | 1 | S | LOW | Y | Y | 1 | 0 | Y |
| 93 | 1 | S | HIGH | N | N | 3 | 130.5 | N |
| 96 | 1 | S | HIGH | N | N | 3 | 67.7 | N |
| 98 | 1 | S | HIGH | N | N | | 64.7 | N |
| 57 | 1 | S | HIGH | N | Y | 3 | 55.4 | N |
| 58 | 1 | S | HIGH | N | Y | | 66.6 | N |
| 94 | 1 | S | HIGH | N | Y | 2 | 0 | Y |
| 85 | 1 | S | HIGH | Y | N | | 0 | Y |
| 99 | 1 | S | HIGH | Y | N | | 1016.1 | N |
| 101 | 1 | S | HIGH | Y | N | | 111.7 | N |
| 83 | 1 | S | HIGH | Y | Y | 3 | 146.8 | N |
| 65 | 1 | S | HIGH | Y | Y | 3 | 0 | Y |
| 103 | 1 | S | HIGH | Y | Y | | 197.6 | N |
| 3 | 3 | S | NONE | N | N | | 156.2 | N |
| 4 | 3 | S | NONE | N | N | | 628 | N |
| 1 | 3 | S | LOW | N | N | | 78.37 | N |
| 2 | 3 | S | LOW | N | N | | 614.5 | N |
| 86 | 3 | S | LOW | N | N | | 78 | N |
| 13 | 3 | S | LOW | N | Y | | 295.7 | N |
| 14 | 3 | S | LOW | N | Y | | 1032.8 | N |
| 85 | 3 | S | LOW | N | Y | 3 | 840.4 | N |
| 5 | 3 | S | LOW | Y | N | | 137.3 | N |
| 6 | 3 | S | LOW | Y | N | | 377.1 | N |
| 90 | 3 | S | LOW | Y | N | | 1016.1 | N |
| 15 | 3 | S | LOW | Y | Y | | 844 | N |
| 16 | 3 | S | LOW | Y | Y | | 577.9 | N |
| 79 | 3 | S | LOW | Y | Y | 2 ID | 8.9 | N |
| 33 | 3 | S | HIGH | N | N | | 711.4 | N |
| 34 | 3 | S | HIGH | N | N | | 1128.7 | N |
| 87 | 3 | S | HIGH | N | N | | 1003.3 | N |
| 37 | 3 | S | HIGH | N | Y | | 642.1 | N |
| 38 | 3 | S | HIGH | N | Y | | 863.5 | N |
| 75 | 3 | S | HIGH | N | Y | 2 | 68.7 | Y |
| 35 | 3 | S | HIGH | Y | N | | 935.3 | N |
| 36 | 3 | S | HIGH | Y | N | | 1235.3 | N |
| 83 | 3 | S | HIGH | Y | N | 2 SC/ID | 219 | N |
| 39 | 3 | S | HIGH | Y | Y | | 804.7 | N |
| 40 | 3 | S | HIGH | Y | Y | | 1315.7 | N |
| 78 | 3 | S | HIGH | Y | Y | 3 | 258.8 | N |

The following is a description of a further experiment demonstrating the importance of dermal-access member velocity. The tests were conducted to determine the more effective dermal-access member seating arrangement between a side push microinfuser and a drop-center infuser. The drop-center manifold (“heavy”) weighs about 7.8 grams, and the side push manifold weights about 0.4-0.6 g. Therefore, for a given spring or spring set used to drive the manifold, the drop-center design will be at least 10 times slower in its initial velocity than the side push design. For this experiment, manifolds weighing about 1.7 grams were used as “light” manifolds. The results are shown in Table 3. For the 3 mm dermal-access members, the light manifolds had an average flow rate of about 3 times than that of the heavy manifolds. This indicates that for the 3 mm needles, the heavy manifold seated the needles to a considerably shallower depth than the light manifold. This is because shallower infusions are known to have a higher back pressure than deeper infusions. The differences shown in the 1 mm dermal-access members were even greater, and none of the heavier 1 mm manifolds were successfully seated.

TABLE 3
MANIFOLD WEIGHT SUMMARY SHEET: 150CT02
CONSTANT PRESSURE: 15 PSI; 50 uL AIR BOLUS

| | | | | | |
|---|---|---|---|---|---|
| | | | BLEB | AVG | |
| TYPE | EXP | LEAKER | TYPE | RATE | IF NO LEAKS |

HEAVY MANIFOLD 3 mm NEEDLE

| | | | | | |
|---|---|---|---|---|---|
| HM3 | 3 | N | 3 ID | 5670 | 5670 |
| HM3 | 6 | N | 1-2 ID | 402 | 402 |
| HM3 | 12 | N | 3 ID | 1000 | 1000 |
| HM3 | 14 | N | ID-SC | 19500 | 19500 |
| | | | AVERAGE | 6643 | 6643 |
| | | | STDEV | 8889 | 8888.995219 |

LIGHT MANIFOLD 3 mm NEEDLE

| | | | | | |
|---|---|---|---|---|---|
| LM3 | 7 | N | ID/SC | 31700 | 31700 |
| LM3 | 9 | N | 1 ID | 962 | 962 |
| LM3 | 10 | N | 2-3 ID | 2330 | 2330 |
| LM3 | 16 | N | SC | 64700 | 64700 |
| LM3 | 17 | N | ID/SC | 18600 | 18600 |
| LM3 | 19 | N | SC | 47300 | 47300 |
| | | | AVERAGE | 27598.7 | 27598.66667 |
| | | | STDEV | 25339.6 | 25339.55774 |

HEAVY MANIFOLD 1 mm NEEDLE

| | | | | | |
|---|---|---|---|---|---|
| HM1 | 1 | Y | 1 TINY | N/A | N/A |
| HM1 | 5 | Y | NONE | N/A | N/A |
| HM1 | 8 | Y | NONE | N/A | N/A |
| HM1 | 11 | Y | NONE | N/A | N/A |
| | | | AVERAGE | 0 | N/A |
| | | | STDEV | N/A | N/A |

LIGHT MANIFOLD 1 mm NEEDLE

| | | | | | |
|---|---|---|---|---|---|
| LM1 | 2 | N | 3 ID | 3990 | 3990 |
| LM1 | 4 | N | 3 ID | 9440 | 9440 |
| LM1 | 13 | N | 3 ID | 12250 | 12250 |
| LM1 | 15 | Y | 2 SMALL | 0 | |
| | | | ID | | |
| LM1 | 18 | Y | 3 SMALL | 0 | |
| | | | ID | | |
| | | | AVERAGE | 5136 | 8560 |
| | | | STDEV | 5549.86 | 4199.726182 |

The lack of obstructions on the face of the device has also been shown to increase effective dermal-access member seating. For example, the exemplary embodiment shown in FIG. 30 has a single surface, i.e., without the raised or recessed first or second surface areas discussed in previous embodiments. The effectiveness of needle seating for an obstructionless device face was shown in a further experiment. The device of FIG. 30 was incorporated into a mechanical applicator for applying the device to a subject at a constant pressure, constant volume, constant dermal-access member length and constant overtravel amount. The leakage rates for these trials were compared to those of trials using a device identical to that shown in FIG. 30, except that the device had walls extending around the periphery of the bottom face of the device, flush with the walls of the parallepiped shaped and at a height equal to that of the tops of the protrusions. The device with the walls leaked more often than the device without walls. It was determined that the presence of a wall on the device only hurts infusion reliability. It is believed that the wall limits the amount of overtravel of the device, and further, prevents the skin in the immediate proximity of the protrusions from wrapping around the protrusions. This agrees with the results of the experiment depicted in Table 1 and discussed above.

While various embodiments have been chosen to illustrate the invention, it will be appreciated by those skilled in the art that various additions and modifications can be made to the invention without departing from the scope of the invention as defined in the appended claims. For example, the body of the device may be made as an integral one-piece unit. In alternative embodiments, the body can be made from separately molded sections or pieces and assembled together. The molded sections can be assembled using an adhesive, by welding, or by the use of mechanical fasteners. Additionally, any number of dermal-access members may be provided on the device.

We claim:

1. An intradermal injection device, comprising:
a chamber configured for containing a substance to be injected;
a needle operatively associated with the chamber and having a length sufficient to deliver the substance to an intradermal injection site; and
a collar surrounding the needle and defining a collar cavity, the collar having a peripheral forward skin-contacting surface that surrounds and is radially spaced from the needle and injection site by an area that is sufficiently large to allow a patient's skin to move into the collar cavity to properly position the needle for intradermal delivery of the substance to the injection site to allow spread of the injected substance under the skin while inhibiting or preventing backpressure within the skin from forcing the substance out through the injection site.

2. The injection device of claim 1, further comprising an energy source associated with the needle to assist in delivering the substance to the injection site.

3. The injection device of claim 2, wherein the energy source is configured to provide an injection assisting pressure of between about 50 and 300 psi to the substance.

4. The injection device of claim 1, wherein the collar has a circular peripheral surface and an internal diameter of about 4 mm to 7 mm.

5. The injection device of claim 1, wherein the skin-contacting surface of the collar is discontinuous.

6. The injection device of claim 5, wherein the skin-contacting surface defines discontinuity gaps, the skin-contacting surface and gaps together defining a closed shape about the needle, wherein the skin-contacting surface occupies at least about 50% of the closed shape.

7. The injecting device of claim 6, wherein the closed shape is rounded.

8. The injection device of claim 7, closed shape is circular.

9. The injection device of claim 5, wherein the discontinuity gaps are substantially equally spaced along the shape.

10. The injection device of claim 5, wherein the skin-contacting surface has at least two substantially equally sized continuous portions separated by the discontinuity gaps.

11. The injection device of claim 1, wherein the needle has a delivery end having a position in which it is disposed within about 0.5 mm of the forward skin-contacting surface.

12. The injection device of claim 1, wherein the needle and collar are configured for injecting the substance intradermally.

13. The injection device as of claim 1, further comprising a central protrusion wherein said needle extends from the protrusion, said protrusion having a height ranging from 0.5 to 1.0 mm, said profusion terminating at a distal free end, said proximal free end defining a width ranging from 0.35 mm to 0. 6 mm.

14. The injection device of claim 1, wherein the collar includes three raised protrusions spaced at 120° angles from one another, radially equidistant from the center of the bottom face.

15. A method of injecting a substance intradermally, which comprises delivering a substance to an intradermal injection site through a needle while contacting the skin with a surface that is spaced from the needle by an area surrounding the needle and injection site, which area is sufficiently large to allow the substance to be intradermally injected to allow spread of the injected substance under the skin while inhibiting or preventing backpressure within the skin from forcing the substance out through the injection.

16. The method of claim 15, further comprising assisting the delivery of the substance to the intradermal injection site by applying a pressure to the substance.

17. The method of claim 16, wherein the pressure is about between 50 and 300 psi.

18. The method of claim 15, wherein the skin-contacting surface comprises a collar that is part of an injection device, wherein the collar has a circular peripheral surface and an internal diameter of about 4 mm to 7 mm.

19. The method of claim 18, wherein the skin is contacted by the skin-contacting surface at contact locations separated by discontinuity gaps, the contact locations and the gaps disposed along a substantially closed shape, and the surface at the contact locations occupying at least about 50% of the closed shape.

20. The method of claim 19, wherein the discontinuity gaps are substantially equally spaced from each other.

21. The method of claim 18, wherein the collar has at least two substantially equally sized continuous portions.

22. The method of claim 16, wherein the needle has a length which sticks out beyond the collar surface by a distance of up to 0.5 mm.
